SEQUENCE LISTING (5 sequences)

SEQ ID NO: 1 - protein, 25 aa, synthetic construct
MAADIISTIGDLVKWIIDTVNKFKK

SEQ ID NO: 2 - DNA, 38 bp, synthetic construct
  source 1..38: mol_type = other DNA
cttacattaattgcgttgcgttgacggctagctcagtc

SEQ ID NO: 3 - DNA, 45 bp, synthetic construct
  source 1..45: mol_type = other DNA
gataccggtaggctgcatttttttaaatttgttcacggtatcaat

SEQ ID NO: 4 - DNA, 195 bp, synthetic construct
  source 1..195: mol_type = other DNA
ttgacggctagctcagtcctaggtacagtgctagctactagagaaagaggagaaatactgatgaaaaagattaccgctgctgctggtctgctgctcctcgctgcccagccggcgatggcgatggcggcggatattattagcaccattggcgatctggtgaaatggattattgataccgtgaacaaatttaaaaaa

SEQ ID NO: 5 - DNA, 217 bp, synthetic construct
  source 1..217: mol_type = other DNA
ttgacggctagctcagtcctaggtacagtgctagctactagagaaagaggagaaatactagatgaagtacctgctgccgaccgcggcggcgggtctgctgctgctggcggcgcagccggcgatggcggacgatgacgatgacatggcggcggatattattagcaccattggcgatctggtgaaatggattattgataccgtgaacaaatttaaaaaa

(19) United States
(12) Patent Application Publication
(10) Pub. No.: US 2026/0218210 A1
(43) Pub. Date: Jul. 30, 2026

(54) ENGINEERED BACTERIA FOR CELLULAR BARRIER MAINTENANCE AND PROTECTION

(71) Applicant: The Trustees of Columbia University in the City of New York, New York, NY (US)

(72) Inventors: Harris He Wang, New York, NY (US); Carlotta Ronda, New York, NY (US); Leonie Brockmann, New York, NY (US); Logan Schwanz, New York, NY (US)

(21) Appl. No.: 19/648,531

(22) Filed: Apr. 15, 2026

Related U.S. Application Data
(63) Continuation of application No. PCT/US2024/051612, filed on Oct. 16, 2024.
(60) Provisional application No. 63/590,666, filed on Oct. 16, 2023.

(51) Int. Cl. C12N 15/70; A61K 35/00; A61K 38/00; A61P 1/00; A61P 1/04; A61P 3/08; A61P 29/00; C07K 14/31; C12N 1/20; C12R 1/19

(52) U.S. Cl. CPC C12N 15/70; A61P 1/00; A61P 1/04; A61P 3/08; A61P 29/00; C07K 14/31; C12N 1/20; A61K 2035/115; A61K 38/00; C12R 2001/19

(57) ABSTRACT

The present disclosure relates to engineering bacteria and methods for promoting barrier function in skin and mucosal surfaces. In particular, the disclosure is related to engineering bacterium to produce a delta hemolysin (delta toxin) peptide and methods of using the engineered bacteria or the delta hemolysin peptide for improving or maintaining gut health, regulating glucose metabolism, maintaining intestinal integrity, decreasing inflammation, modulating wound healing and tissue regeneration, and treating or preventing a disease or disorder.

### FIELD

The present disclosure relates to engineering bacteria and methods for promoting barrier function in skin and mucosal surfaces. In particular, the disclosure is related to engineering bacterium to produce a delta hemolysin (delta toxin) peptide and methods of using the engineered bacteria or the delta hemolysin peptide for improving or maintaining gut health, regulating glucose metabolism, maintaining intestinal integrity, decreasing inflammation, modulating wound healing and tissue regeneration, and treating or preventing a disease or disorder.

### CROSS-REFERENCE TO RELATED APPLICATIONS

This application is a continuation of PCT International Application No. PCT/US2024/051612, filed Oct. 16, 2024, which claims the benefit of U.S. Provisional Application No. 63/590,666, filed Oct. 16, 2023, the contents of which are herein incorporated by reference in its entirety.

### SEQUENCE LISTING STATEMENT

The content of the electronic sequence listing titled COLUM_42482.601.xml (Size: 5,890 bytes; and Date of Creation: Oct. 16, 2024) is herein incorporated by reference in its entirety.

### BACKGROUND

Inflammatory Bowel Disease (IBD) is a broad term that describes conditions characterized by chronic inflammation of the gastrointestinal tract. The two most common inflammatory bowel diseases are Crohn's disease and ulcerative colitis. IBD is a chronic condition with symptoms that tend to wax and wane with frequent exacerbations. Besides IBD, other chronic colon diseases, such as irritable bowel syndrome, similarly require long-term management. The escalation in the incidence of IBD in western nations encompasses contributions from genetic susceptibility and pivotal environmental determinants, including diet and lifestyle factors such as smoking and alcohol consumption. A characteristic western diet, high in fats and sugars yet low in fiber, engenders significant alterations in gut microbiota, precipitating dysbiosis and notably impacting gut permeability and susceptibility to colitis through the alteration of bacterial communities. While fiber supplementation can augment gut barrier function and deter colitis development, its application is constrained for IBD patients due to associated symptoms and potential side effects such as abdominal pain and constipation. Thus, additional compositions, systems and methods are needed to manage and treat chronic diseases.

### SUMMARY

Provided herein are bacterium engineered for the production of a heterologous delta hemolysin peptide or variant or polypeptide comprising thereof. In some embodiments, the bacterium comprises a nucleic acid encoding the delta hemolysin peptide. In some embodiments, the nucleic acid is incorporated into the host genome. In some embodiments, the nucleic acid is episomal.

In some embodiments, the delta hemolysin peptide is derived from a Staphylococcal species. In some embodiments, the delta hemolysin peptide is derived from Staphylococcus epidermidis. In some embodiments, the delta hemolysin peptide has an amino acid sequence of MAADIISTIGDL VKWIIDTVNKFKK (SEQ ID NO: 1).

In some embodiments, the delta hemolysin peptide is linked to a signal peptide. In some embodiments, the signal peptide is a NSP4 secretion tag/signal peptide.

In some embodiments, the bacterium is a non-pathogenic bacterium. In some embodiments, the bacterium is a commensal bacterium. In some embodiments, the bacterium is a probiotic bacterium. In some embodiments, the bacterium is from the genus Escherichia. In some embodiments, the bacterium is Escherichia coli strain Nissle. In some embodiments, the bacterium is isolated from a subject. In some embodiments, the bacterium is a strain of gut bacteria.

Also provided herein are compositions comprising a bacterium as disclosed herein, or a population thereof. In some embodiments, the composition further comprises a pharmaceutically acceptable carrier or excipient. In some embodiments, the composition is a food or beverage product. In some embodiments, the compositions is formulated for oral or rectal administration.

Further provided are methods for improving or maintaining gut health, regulating glucose metabolism, maintaining intestinal integrity, and/or promoting tissue repair and regeneration.

In some embodiments, the methods comprise administering to a subject in need thereof a bacterium as disclosed herein, or a population or composition comprising thereof. In some embodiments, the administration comprises oral administration, rectal administration, intravenous injection, nasal administration, and subcutaneous injection.

In some embodiments, the methods comprise introducing a nucleic acid encoding a delta hemolysin peptide or variant or polypeptide into a bacterium in the subject.

In some embodiments, the delta hemolysin peptide is derived from a Staphylococcal species. In some embodiments, the delta hemolysin peptide is derived from Staphylococcus epidermidis. In some embodiments, the delta hemolysin peptide has an amino acid sequence of MAADIISTIGDLVKWIIDTVNKFKK (SEQ ID NO: 1). In some embodiments, the delta hemolysin peptide is linked to a signal peptide. In some embodiments, the signal peptide is a NSP4 secretion tag/signal peptide.

In some embodiments, the subject or is suspected of having has a disease or disorder. In some embodiments, the disease or disorder is a gastrointestinal disease or disorder or an inflammatory disease or disorder. In some embodiments, the subject has or is suspected of having inflammatory bowel disease. In some embodiments, the subject has or is suspected of having Crohn's disease and ulcerative colitis.

Other aspects and embodiments of the disclosure will be apparent in light of the following detailed description.

### BRIEF DESCRIPTION OF THE DRAWINGS

FIG. 1 is a schematic of an exemplary vector used in production of peptides in bacteria.

FIGS. 2A-2C show that Nissle Hld_{SE} promotes barrier function in WT mice fed no fiber diet (NFD). FIG. 2A is a schematic of an exemplary experimental design. FIG. 2B is a graph of weight loss of normal chow (NC) and no fiber diet (NFD) fed mice. E. coli Nissle (EcN) or E. coli Nissle engineered to produce delta-hemolysin peptide (EcN Hld_{SE}) (10\hat{ }10 CFU/mouse) was gavaged every other day for five weeks. N=3-4 mice/group, one experiment. FIG. 2C is a graph of colon length of NC and NFD fed mice. EcN or EcN Hld_{SE} (10\hat{ }10 CFU/mouse) was gavaged every other day for five weeks. N=3-4 mice/group, one experiment.

FIGS. 3A-3H show Nissle Hld_{SE} protects from acute dextran sodium sulfate (DSS) colitis by promoting gut barrier function. FIG. 3A is a schematic of an exemplary experimental design. FIGS. 3B and 3C are graphs of colon length (FIG. 3B) and liver CFU (FIG. 3C) after 6 days of DSS treatment of NFD fed mice. EcN or EcN Hld_{SE} (5×10\hat{ }9 CFU/mouse) was gavaged daily starting three days prior to DSS administration. N=18-23 mice/group, four independent experiments. FIGS. 3D and 3E are graphs of weight loss (FIG. 3D) and survival (FIG. 3E) during the course of DSS treatment of NFD fed mice. EcN or EcN Hld_{SE} (5×10\hat{ }9 CFU/mouse) was gavaged daily starting three days prior to DSS administration. N=18-23 mice/group, four independent experiments. FIG. 3F is a schematic of an exemplary experimental design. FIG. 3G is a graph of colon length after 6 days of DSS treatment of NC fed mice. EcN or EcN Hld_{SE} (10\hat{ }10 CFU/mouse) were gavaged daily starting three days prior to DSS administration. N=5 mice/group, one experiment. FIG. 3H is a graph of colon length after 6 days of peptide treatment of NC fed mice. EcN or EcN Hld_{SE} (10\hat{ }10 CFU/mouse) were gavaged daily. N=5 mice/group, one experiment.

FIGS. 4A-4C show the protective effect of Nissle Hld_{SE} is dose dependent. FIG. 4A is a schematic of an exemplary experimental design. FIG. 4B is a graph of weight loss during the course of DSS treatment of NFD fed mice. EcN Hld_{SE} (1×10\hat{ }8; 5×10\hat{ }9 or 1×10\hat{ }10 CFU/mouse) was gavaged daily starting three days prior to DSS administration. N=4-5 mice/group, one experiment. FIG. 4C is a graph of colon length after 5 days of DSS treatment of NFD fed mice. EcN Hld_{SE} (1×10\hat{ }8; 5×10\hat{ }9 or 1×10\hat{ }10 CFU/mouse) was gavaged daily starting three days prior to DSS administration. N=4-5 mice/group, one experiment.

FIGS. 5A-5C show Nissle Hld_{SE} promotes gut barrier function in Il10^{-/-} mice, a chronic colitis model. FIG. 5A is a schematic of an exemplary experimental design. FIG. 5B is a graph of weight loss of NFD fed Il10^{-/-} mice. EcN or EcN Hld_{SE} (1×10\hat{ }10 CFU/mouse) was gavaged every other day for five weeks. N=6-7 mice/group, two independent experiments. FIG. 5C is a graph of colon length of NFD fed I110^{-/-} mice after five weeks of diet intervention. EcN or EcN Hld_{SE} (1×10\hat{ }10 CFU/mouse) was gavaged every other day. N=6-7 mice/group, two independent experiments.

FIG. 6 is a graph of the frequency of IL-17A⁺ cells within IEL γδ (gd) T cells after 6 days of DSS treatment of NFD fed mice.

FIGS. 7A-7D show Nissle Hld_{SE} promotes gut barrier function in part via IEL γδ (gd) T cells. FIG. 7A is a schematic of an exemplary experimental design. FIG. 7B is a graph of the frequency of IEL gd T cells after 6 days of DSS treatment of NFD fed mice. EcN or EcN Hld_{SE} (5×10\hat{ }9 CFU/mouse) was gavaged daily starting three days prior to DSS administration. gd T cells were depleted by administration of neutralizing antibodies i.p. every other day. FIG. 7C is a graph of weight loss during the course of DSS treatment of NFD fed mice. EcN or EcN Hld_{SE} (5×10\hat{ }9 CFU/mouse) was gavaged daily starting three days prior to DSS administration. gd T cells were depleted by administration of neutralizing antibodies i.p. every other day. N=3-5 mice/group, one experiment. FIG. 7D is a graph of colon length after 6 days of DSS treatment of NED fed mice. EcN or EcN Hld_{SE} (5×10\hat{ }9 CFU/mouse) was gavaged daily starting three days prior to DSS administration. gd T cells were depleted by administration of neutralizing antibodies i.p. every other day. N=3-5 mice/group, one experiment.

FIGS. 8A-8K show dietary fiber deficiency disrupts intestinal immune and enteroendocrine homeostasis. FIG. 8A is a schematic of dietary intervention under steady state condition. FIG. 8B is a graph of weight development of mice fed normal diet (ND) or fiber-deficient diet (FDD) for five weeks. Cumulative of two independent experiment, n=6-8 mice/group. FIG. 8C is a graph of colon length after feeding of ND or FDD for five weeks. Cumulative of four independent experiment, n=17-20 mice/group. FIG. 8D is a graph of isolated lymphoid follicle (ILF) count in the SI and LI of ND and FDD mice after 5 weeks of dietary intervention. Cumulative of two independent experiments, n=9 mice/group. FIG. 8E is a heatmap of differentially expressed genes (DEGs) from RNA-sequencing of colon from mice fed ND or FDD for five weeks. One experiment, n=4 mice/group, top KEGG and GO: BP pathways in over-representation analysis on the right and example DEGs on the left. FIG. 8F is principal component analysis of 16S sequencing of fecal samples from ND and FDD mice after one week and 3 weeks of dietary intervention. FIG. 8G is a plot of bacterial richness of fecal samples from ND and FDD mice after zero, one and three weeks of dietary intervention. One experiment, n=4-5 mice/group. FIG. 8H is a graph of differential abundant species based on the 16S sequencing in (FIG. 8F). FIG. 8I shows results from quantitative PCR for selected L-cell related genes from colon of mice fed ND or FDD for five weeks. Cumulative of three independent experiments, n=14-16 mice/group. FIG. 8J is a graph of number of L-cells based on GLP-1 staining in colon of mice fed ND or FDD for five weeks. One independent experiment, n=5 mice/group; 10 fields/colon. FIG. 8K is a graph of the concentration of GLP-1 in plasma of mice fed ND or FDD for five weeks. Cumulative of two independent experiments, n=5-6 mice/group. Values normalized to average of ND fed mice in each experiment.

FIGS. 9A-9L show that the increase of GLP-1 release in response to tissue damage is stunted under fiber deficient condition. FIG. 9A is a graph of weight development of ND mice during five days of DSS colitis with indicated DSS concentrations. Cumulative of two independent experiments, n=3-7 mice/group. FIG. 9B is a graph of colon length of ND mice after five days of DSS colitis with indicated DSS concentrations. Cumulative of two independent experiments, n=3-7 mice/group. FIG. 9C is a graph of concentration of GLP-1 in plasma of ND mice after five days of DSS colitis with indicated DSS concentrations. Cumulative of two independent experiments, n=3-7 mice/group. FIG. 9D is a graph showing the correlation between plasma GLP-1 and colon length of ND mice after five days of DSS colitis with indicated DSS concentrations. Cumulative of two independent experiments, n=20 mice. FIG. 9E is a schematic of ND mice under DSS colitis condition with additional treatment with GLP-1 receptor antagonist. FIG. 9F is a graph of weight development of ND mice during five days of DSS colitis with or without treatment with GLP-1 receptor antagonist. Cumulative of two independent experiments, n=10 mice/group. FIG. 9G is a graph of colon length of ND mice after five days of DSS colitis with or without treatment with GLP-1 receptor antagonist. Cumulative of two independent experiments, n=10 mice/group. FIG. 9H is a graph of colony forming units (CFU) in liver of ND mice after five days of DSS colitis with or without treatment with GLP-1 receptor antagonist. Cumulative of two independent experiments, n=10 mice/group. FIG. 9I is a graph of weight development of FDD mice during five days of DSS colitis with indicated DSS concentrations. Cumulative of two independent experiments, n=4-6 mice/group. FIG. 9J is a graph of colon length of FDD mice after five days of DSS colitis with indicated DSS concentrations. Cumulative of two independent experiments, n=4-5 mice/group. FIG. 9K is a graph of CFU in liver of FDD and ND mice after five days of DSS colitis. Cumulative of two independent experiments, n=3-7 mice/group. FIG. 9L is a graph of concentration of GLP-1 in plasma of FDD and ND mice after five days of DSS colitis with indicated DSS concentrations. Cumulative of two independent experiments, n=3-6 mice/group.

FIGS. 10A-10I show that a GLP-1 receptor agonist can ameliorate severe colitis in dysbiotic mice. FIG. 10A is a schematic of DSS colitis conditions with GLP-1 receptor agonist treatment in FDD mice. FIG. 10B is a graph of weight development of FDD mice during five days of DSS colitis with or without GLP-1 receptor agonist treatment. Cumulative of three independent experiments, n=16-17 mice/group. FIG. 10C is a graph of colon length of FDD mice after five days of DSS colitis with or without GLP-1 receptor agonist treatment. Cumulative of three independent experiments, n=14 mice/group. FIG. 10D is a graph of CFU in liver of FDD mice after five days of DSS colitis with or without GLP-1 receptor agonist treatment. Cumulative of three independent experiments, n=14 mice/group. FIG. 10E shows the concentration of plasma TNF-α, IL-1α, IL-6 and MCP-1 of FDD mice after five days of DSS colitis with or without GLP-1 receptor agonist treatment. Cumulative of two independent experiments, n=8 mice/group. FIG. 10F shows gene set enrichment analysis (GSEA) of RNA-sequencing samples from colon of FDD mice after five days of DSS colitis with or without GLP-1 receptor agonist treatment with modules (gene lists) identified in DSS colitis time-series (Czarnewski, P., et al., Nat Commun, 2019. 10 (1): p. 2892). One experiment, n=3 mice/group. FIG. 10G is a heatmap of DEGs from RNA-sequencing samples in FIG. 10F, top KEGG and GO: BP pathways in over-representation analysis on the right and example DEGs on the left. FIG. 10H shows enriched GO: CC term ‘Apical junction complex’ in GSEA from RNA-sequencing samples in FIG. 10F with leading edge gene expression on the right. FIG. 10I shows enriched GO: BP term ‘Maintenance of gastrointestinal epithelium’ in GSEA from RNA-sequencing samples in FIG. 10F with leading edge gene expression on the right.

FIGS. 11A-11M show the engineered probiotic as described herein improves colitis outcome in dysbiotic mice in GLP-1 dependent manner. FIG. 11A is a schematic of DSS colitis conditions with either EcN or EcN-Hld_{SE} treatment in FDD mice. FIG. 11B is a graph of the concentration of GLP-1 in plasma of FDD mice after five days of DSS colitis with either EcN or EcN-Hld_{SE} treatment. Cumulative of two independent experiments, n=4 mice/group. FIG. 11C is a survival curve of FDD mice after five days of DSS colitis with either EcN or EcN-Hld_{SE} treatment. Cumulative of five independent experiments, n=23-25 mice/group. FIG. 11D is a graph of weight development of FDD mice during five days of DSS colitis with either EcN or EcN-Hld_{SE} treatment. Cumulative of five independent experiments, n=23-25 mice/group. FIG. 11E is a graph of colon length of FDD mice after five days of DSS colitis with either EcN or EcN-Hld_{SE} treatment. Cumulative of five independent experiments, n=17 mice/group. FIG. 11F is a graph of the CFU in liver of FDD mice after five days of DSS colitis with either EcN or EcN-Hld_{SE} treatment. Cumulative of two independent experiments, n=7 mice/group. FIG. 11G shows the concentration of plasma TNF-α, IL-1α, IL-6 and MCP-1 of FDD mice after five days of DSS colitis with either EcN or EcN-Hld_{SE} treatment. Cumulative of three independent experiments, n=8-13 mice/group. FIG. 11H shows the correlation between plasma GLP-1 and colon length of FDD mice after five days of DSS colitis with either EcN or EcN-Hld_{SE} treatment. Cumulative of two independent experiments, n=8 mice. FIG. 11I is a schematic of DSS colitis conditions with EcN-Hld_{SE} treatment with or without injection of GLP-1 receptor antagonist in FDD mice. FIG. 11J is a graph of the weight development of FDD mice during five days of DSS colitis with EcN-Hld_{SE} treatment with or without injection of GLP-1 receptor antagonist. Cumulative of three independent experiments, n=11-15 mice/group. FIG. 11K is a graph of the colon length of FDD mice after five days of DSS colitis with EcN-Hld_{SE} treatment with or without injection of GLP-1 receptor antagonist. Cumulative of three independent experiments, n=8-14 mice/group. FIG. 11L is a graph of the CFU in liver of FDD mice after five days of DSS colitis with EcN-Hld_{SE} treatment with or without injection of GLP-1 receptor antagonist. Cumulative of two independent experiments, n=7-8 mice/group. FIG. 11M shows the concentration of plasma TNF-α, IL-1α, IL-6 and MCP-1 of FDD mice after five days of DSS colitis with EcN-Hld_{SE} treatment with or without injection of GLP-1 receptor antagonist. Cumulative of three independent experiments, n=10-13 mice/group.

FIGS. 12A-12F show that GLP-1 modulates genes involved in core intestinal functions. FIG. 12A shows panther protein classes of up- and downregulated genes with a minimum of 1 average log 2 fold-change from ranked gene list (described in methods). FIG. 12B shows the top 20 most enriched GO: CC pathways and GO: BP pathways in GSEAPreranked based on the ranked gene list of average log 2 fold changes of all genes. FIG. 12C shows enriched GO: BP term ‘Pyroptisis’ in GSEAPreranked with leading edge gene expression on the right.

FIG. 12D is a heatmap of selected upregulated transcription factors and upregulated secretory cell linage related genes. FIG. 12E shows the number of L-cells based on GLP-1 staining, and goblet cells based in Alcian Blue staining, in colon of mice fed FDD with or without treatment with GLP-1 receptor agonist during DSS colitis. One independent experiment, n=5 mice/group; 10 fields/colon. FIG. 12F is a heatmap of selected transcription factors and upregulated secretory cell linage related genes in human colon biopsies.

FIGS. 13A-13C show fiber deficient diet causes disruption of immune and enteroendocrine homeostasis. FIG. 13A shows CD11b and Ly6G expression (neutrophils) in myeloid cells (CD11c⁺ CD11b⁺ TCRb⁻ B220⁻) in colon of mice fed ND or FDD for five weeks. Cumulative of two independent experiments, n=7-8 mice/group. FIG. 13B shows results from quantitative PCR for selected apical junction genes from colon of mice fed ND or FDD for five weeks. Cumulative of three independent experiments, n=10-16 mice/group. FIG. 13C shows representative GLP-1 staining in colon of mice fed ND or FDD for five weeks.

FIGS. 14A-14T show dietary fiber deficiency causes severe colitis. FIG. 14A is a schematic of dietary intervention under DSS colitis conditions. FIG. 14B is a survival curve of mice fed FDD or ND for seven days prior to five days of DSS colitis. Cumulative of three independent experiments, n=13-17 mice/group. FIG. 14C is a graph of weight development of FDD or ND mice during five days of DSS colitis. Cumulative of three independent experiments, n=13-17 mice/group. FIG. 14D is a graph of colon length of FDD or ND mice after five days of DSS colitis. Cumulative of three independent experiments, n=10-13 mice/group. FIG. 14E is a graph of colony forming units (CFU) in liver of FDD or ND mice after five days of DSS colitis. Cumulative of two independent experiments, n=6-7 mice/group. FIG. 14F is a graph of colitis score based on assessment of H&E staining of colon section from FDD or ND mice after five days of DSS colitis. One independent experiment, n=4 mice/group. FIG. 14G shows CD11b and Ly6G expression (neutrophils) in myeloid cells (CD11c⁺ CD11b⁺ TCRb⁻ B220⁻) in colon of mice ND- or FDD fed mice after five days of DSS colitis. Cumulative of two independent experiments, n=6-9 mice/group. FIG. 14H is a schematic of dietary intervention in Il10^{-/-} mice. FIG. 14I is a graph of weight development of Il10^{-/-} mice fed ND or FDD for five weeks. Cumulative of two independent experiment, n=6-8 mice/group. FIG. 14J is a graph of colon length of Il10^{-/-} mice fed ND or FDD for five weeks. Cumulative of two independent experiment, n=6-8 mice/group. FIG. 14K is a graph of CFU in liver of Il10^{-/-} mice fed ND or FDD for five weeks. Cumulative of three independent experiment, n=6-11 mice/group. FIG. 14L is graphs of quantitative PCR for selected apical junction genes from colon of Il10^{-/-} mice fed ND or FDD for five weeks. Cumulative of two independent experiments, n=6-10 mice/group. FIG. 14M shows CD11b and Ly6G expression (neutrophils) in myeloid cells (CD11c⁺ CD11b⁺ TCRb⁻ B220⁻) in colon of mice ND- or FDD fed Il10^{-/-} mice after five weeks of dietary intervention. Cumulative of two independent experiments, n=6-8 mice/group. FIG. 14N is a schematic of dietary intervention under DSS colitis conditions with additional supplementation of butyrate. FIG. 14O is a graph of weight development of FDD mice during five days of DSS colitis with or without butyrate supplementation. Cumulative of two independent experiments, n=10 mice/group. FIG. 14P is a graph of colon length of FDD mice after five days of DSS colitis with or without butyrate supplementation. Cumulative of two independent experiments, n=8-10 mice/group. FIG. 14Q is a graph of CFU in liver of FDD mice after five days of DSS colitis with or without butyrate supplementation. One independent experiment, n=4-5 mice/group. FIG. 14R is a graph of CFU in liver of ND mice after five days of DSS colitis with indicated DSS concentrations. Cumulative of two independent experiments, n=3-7 mice/group. FIG. 14S shows the correlation between plasma GLP-1 and weight of ND mice after five days of DSS colitis with indicated DSS concentrations. Cumulative of two independent experiments, n=20 mice. FIG. 14T shows the correlation between plasma GLP-1 and colon length/weight of FDD mice after five days of DSS colitis with indicated DSS concentrations. Cumulative of two independent experiments, n=17 mice.

FIGS. 15A-15M show an engineered probiotic, as described herein, increases GLP-1 level and promotes intestinal health. FIG. 15A is a graph of the concentration of GLP-1 in plasma of mice fed ND or FDD for one week with daily gavage of 10¹⁰ CFU of either EcN or EcN-Hld_{SE}. Cumulative of two independent experiments, n=4-6 mice/group. FIG. 15B is a schematic of dietary intervention under steady state condition with additional gavage of 10¹⁰ CFU/mouse EcN or EcN-Hld_{SE} every other day for five weeks. FIG. 15C is a graph of colon length after feeding of ND or FDD for five weeks accompanied of gavage of 10¹⁰ CFU/mouse EcN or EcN-Hld_{SE} every other day. Cumulative of two independent experiments, n=9-10 mice/group. FIG. 15D shows CD11b and Ly6G expression (neutrophils) in myeloid cells (CD11c⁺ CD11b⁺ TCRb⁻ B220⁻) in colon of mice fed ND or FDD for five weeks accompanied of gavage of 10¹⁰ CFU/mouse EcN or EcN-Hld_{SE} every other day. Cumulative of two independent experiments, n=5-6 mice/group. FIG. 15E is a schematic of FDD mice under DSS colitis condition with additional gavage of indicated CFU/mouse EcN-Hld_{SE} every day starting two days prior to DSS supplementation. FIG. 15F is a graph of colon length of FDD mice after five days of DSS colitis with additional gavage of indicated CFU/mouse EcN-Hld_{SE} every day. One experiment, n=4-5 mice/group. FIG. 15G is a graph of weight development of FDD mice during five days of DSS colitis with additional gavage of indicated CFU/mouse EcN-Hld_{SE} every day. One experiment, n=4-5 mice/group. FIG. 15H is a schematic of dietary intervention in Il10^{-/-} mice with additional gavage of 10¹⁰ CFU/mouse EcN or EcN-Hld_{SE} every other day for five weeks. FIG. 15I is a graph of weight development of FDD Il10^{-/-} mice with additional gavage of 10¹⁰ CFU/mouse EcN or EcN-Hld_{SE} every other day for five weeks. Cumulative of three independent experiments, n=13-14 mice/group. FIG. 15J is a graph of colon length of FDD I110^{-/-} mice with additional gavage of 10¹⁰ CFU/mouse EcN or EcN-Hld_{SE} every other day for five weeks. Cumulative of three independent experiments, n=13-14 mice/group. FIG. 15K is a graph of CFU in liver of FDD Il10^{-/-} mice with additional gavage of 10¹⁰ CFU/mouse EcN or EcN-Hld_{SE} every other day for five weeks. Cumulative of two independent experiments, n=7-11 mice/group. FIG. 15L shows the concentration of plasma TNF-α, IL-1α, IL-6 and MCP-1 of FDD Il10^{-/-} mice with additional gavage of 10¹⁰ CFU/mouse EcN or EcN-Hld_{SE} every other day for five weeks. Cumulative of three independent experiments, n=11-14 mice/group. FIG. 15M shows the results of quantitative PCR for selected apical junction genes from colon of Il10^{-/-} mice fed FDD with additional gavage of 10¹⁰ CFU/mouse EcN or EcN-Hld_{SE} every other day for five weeks. Cumulative of two independent experiments, n=10-11 mice/group.

FIGS. 16A-16D show transcriptional changes in colon in response to EcN-Hld_{SE}. FIG. 16A is a heatmap of DEGs from RNA-sequencing samples of FDD mice after five days of DSS colitis with either EcN or EcN-Hld_{SE} treatment, top KEGG and GO: BP pathways from over-representation analysis on the right and example DEGs on the left. One experiment, n=3-5 mice/group. FIG. 16B shows enriched GO: CC term ‘apical junction complex’ in GSEA from RNA-sequencing samples in FIG. 16A with leading edge gene expression on the right. FIG. 16C shows enriched GO: BP term ‘epithelial structure maintenance’ in GSEA from RNA-sequencing samples in FIG. 16A with leading edge gene expression on the right. FIG. 16D is a Venn diagram of DEGs from following colon RNA-sequencing comparisons: FDD mice after five days of DSS colitis with EcN gavage and GLP-1 receptor agonist treatment (agonist) versus FDD mice after five days of DSS colitis with EcN gavage (C, control); FDD mice after five days of DSS colitis with EcN-Hld_{SE} gavage (EcN-Hld_{SE}) versus FDD mice after five days of DSS colitis with EcN gavage (C, control).

FIGS. 17A-17C show transcriptional changes in response to GLP-1 modulating therapy in the colon. FIG. 17A shows a Venn diagram of DEGs from following colon RNA-sequencing comparisons: FDD mice after five days of DSS colitis with EcN gavage and GLP-1 receptor agonist treatment (agonist) versus FDD mice after five days of DSS colitis with EcN gavage (C, control); FDD mice after five days of DSS colitis with EcN-Hld_{SE} gavage (EcN-Hld_{SE}) versus FDD mice after five days of DSS colitis with EcN gavage (C, control); FDD mice after five days of DSS colitis with EcN-Hld_{SE} gavage (EcN-Hld_{SE}) versus FDD mice after five days of DSS colitis with EcN-Hld_{SE} gavage and GLP-1 receptor antagonist treatment (EcN-Hld_{SE}+antagonist). One experiment, n=3-5 mice/group. Expression of shared DEGs on the right. FIG. 17B shows GO: CC pathway ‘apical junction complex’ in GSEAP reranked of ranked gene list generated based on RNA-sequencing samples in (FIG. 17A) including expression of heatmap from leading edge genes on the right. FIG. 17C shows GO: BP pathway ‘epithelial structure maintenance’ in GSEAP reranked of ranked gene list generated based on RNA-sequencing samples in FIG. 17A including expression of heatmap from leading edge genes on the right.

### DETAILED DESCRIPTION

The disclosed bacterium, systems, and compositions advance methods for improving or maintaining gut health, regulating glucose metabolism, maintaining intestinal integrity, and promoting tissue repair and regeneration, e.g., against severe inflammatory responses such as in IBD. Utilizing Escherichia coli Nissle 1917 (EcN) engineered to produce delta hemolysin, also referred to as delta-toxin or delta-lysin, derived from Staphylococcus epidermidis (Hld_{SE}), gut integrity was boosted through a targeted probiotic approach. Both in acute (DSS) and chronic (IL 10^{-/-}) murine models, the delta toxin peptide significantly reduced gut inflammation and improved colitis outcomes. An upregulation of IL-17A production by γδ T cells was observed following administration of the probiotic, highlighting a potential mechanism for improved intestinal integrity and function through the promotion of wound healing by γδ T cells.

Section headings as used in this section and the entire disclosure herein are merely for organizational purposes and are not intended to be limiting.

### Definitions

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of,” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

As used herein, the term “bacteria” encompasses both prokaryotic organisms and archaea present in microbiota of a subject or occurring in a natural environment not necessarily within a subject.

The terms “intestinal microbiota,” “gut flora,” “gastrointestinal microbiota,” and the like are used interchangeably to refer to bacteria in the digestive tract.

As used herein, “genetically modified” refers to an organism (e.g., a bacterium) which has a modification to introduce a nucleic acid that does not naturally occur in the organism or to introduce additional copies or modified forms of nucleic acids that naturally occur in the organism. The nucleic acid can be integrated in one or more copies into a genome or one or more copies of the nucleic acid can remain episomal, e.g., in a plasmid, phagemid or artificial chromosome.

The terms “non-naturally occurring,” “engineered,” and “synthetic” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

As used herein, “nucleic acid” or “nucleic acid sequence” refers to a polymer or oligomer of pyrimidine and/or purine bases, preferably cytosine, thymine, and uracil, and adenine and guanine, respectively (See Albert L. Lehninger, Principles of Biochemistry, at 793-800 (Worth Pub. 1982)). The present technology contemplates any deoxyribonucleotide, ribonucleotide, or peptide nucleic acid component, and any chemical variants thereof, such as methylated, hydroxymethylated, or glycosylated forms of these bases, and the like. The polymers or oligomers may be heterogenous or homogenous in composition and may be isolated from naturally occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states. In some embodiments, a nucleic acid or nucleic acid sequence comprises other kinds of nucleic acid structures such as, for instance, a DNA/RNA helix, peptide nucleic acid (PNA), morpholino nucleic acid (see, e.g., Braasch and Corey, Biochemistry, 41 (14): 4503-4510 (2002)) and U.S. Pat. No. 5,034,506), locked nucleic acid (LNA; see Wahlestedt et al., Proc. Natl. Acad. Sci. U.S.A., 97:5633-5638 (2000)), cyclohexenyl nucleic acids (see Wang, J. Am. Chem. Soc., 122:8595-8602 (2000)), and/or a ribozyme. Hence, the term “nucleic acid” or “nucleic acid sequence” may also encompass a chain comprising non-natural nucleotides, modified nucleotides, and/or non-nucleotide building blocks that can exhibit the same function as natural nucleotides (e.g., “nucleotide analogs”); further, the term “nucleic acid sequence” as used herein refers to an oligonucleotide, nucleotide or polynucleotide, and fragments or portions thereof, and to DNA or RNA of genomic or synthetic origin, which may be single or double-stranded, and represent the sense or antisense strand. The terms “nucleic acid,” “polynucleotide,” “nucleotide sequence,” and “oligonucleotide” are used interchangeably. They refer to a polymeric form of nucleotides of any length, either deoxyribonucleotides or ribonucleotides, or analogs thereof.

A “vector” or “expression vector” is a replicon, such as plasmid, phage, virus, or cosmid, to which another DNA segment, e.g., an “insert,” may be attached or incorporated so as to bring about the replication of the attached segment in a cell.

The term “gene” refers to a nucleic acid (e.g., DNA or RNA) sequence that comprises coding sequences necessary for the production of an RNA, or of a polypeptide or its precursor. A functional polypeptide can be encoded by a full-length coding sequence or by any portion of the coding sequence as long as the desired activity or functional properties (e.g., enzymatic activity, ligand binding, signal transduction, etc.) of the polypeptide are retained. The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located in the 5′ of the coding regions and that are present on the mRNA are referred to as 5′ non-translated or untranslated sequences. The sequences that are located 3′ or downstream of the coding region and that are present on the mRNA are referred to as 3′ non-translated or 3′ untranslated sequences.

As used herein, “heterologous” in reference to a gene, peptide, protein, or nucleic acid refers to a gene, peptide, protein, or nucleic acid that is not normally found in a given cell in nature. As used herein, heterologous may encompass a gene, peptide, protein, or nucleic acid that is exogenously introduced into a given cell, while being naturally found or expressed in the cell not naturally found or expressed in the cell.

As used herein, the terms “providing,” “administering,” and “introducing,” are used interchangeably herein and refer to the placement into a subject by a method or route which results in at least partial localization to a desired site. The administration can be by any appropriate route which results in delivery to a desired location in the subject.

As used herein, “treat,” “treating” and the like means a slowing, stopping, or reversing of progression of a disease or disorder when provided a composition described herein to an appropriate control subject. The term also means a reversing of the progression of such a disease or disorder to a point of eliminating or greatly reducing symptoms of the disease. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, subject may include either adults or juveniles (e.g., children). Moreover, patient may mean any living organism, preferably a mammal (e.g., human or non-human). Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment of the methods provided herein, the subject is a human.

The term “contacting” as used herein refers to bring or put in contact, to be in or come into contact. The term “contact” as used herein refers to a state or condition of touching or of immediate or local proximity.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### Engineered Bacteria

The present disclosure provides bacteria engineered for production of a peptide to modulate GLP1 activation in the gut. In some embodiments, the peptide is capable of causing the upregulation of IL-17A production in a cell. In some embodiments, the peptide is a delta hemolysin peptide, also referred to herein as delta toxin.

Accordingly, the disclosure provides bacteria configured for the production of a heterologous delta hemolysin peptide or variant or polypeptide comprising thereof. Delta hemolysin or toxin is a 26 amino acid peptide encoded by the hld gene. In some embodiments, the engineered bacteria comprise a nucleic acid encoding the heterologous delta hemolysin peptide. In some embodiments, the nucleic acid is integrated into the bacterial genome. In some embodiments, the nucleic acid is episomal, e.g., in a plasmid, phagemid or artificial chromosome.

Hemolysins are some of the virulence factors of coagulase-positive (CPS) and coagulase-negative staphylococci (CNS). The delta hemolysin may be derived from any Staphylococcal species. In select embodiments, the delta hemolysin peptide is derived from Staphylococcus epidermidis.

In some embodiments, the delta hemolysin peptide has an amino acid sequence of MAADIISTIGDLVKWIIDTVNKFKK (SEQ ID NO: 1). In some embodiments, the delta hemolysin peptide has one or more (e.g., 1, 2, 3, 4, 5) amino acid substitutions compared to SEQ ID NO: 1.

An amino acid “replacement” or “substitution” refers to the replacement of one amino acid at a given position or residue by another amino acid at the same position or residue within a polypeptide sequence. Amino acids are broadly grouped as “aromatic” or “aliphatic.” An aromatic amino acid includes an aromatic ring. Examples of “aromatic” amino acids include histidine (H or His), phenylalanine (F or Phe), tyrosine (Y or Tyr), and tryptophan (W or Trp). Non-aromatic amino acids are broadly grouped as “aliphatic.” Examples of “aliphatic” amino acids include glycine (G or Gly), alanine (A or Ala), valine (V or Val), leucine (L or Leu), isoleucine (I or He), methionine (M or Met), serine (S or Ser), threonine (T or Thr), cysteine (C or Cys), proline (P or Pro), glutamic acid (E or Glu), aspartic acid (A or Asp), asparagine (N or Asn), glutamine (Q or Gin), lysine (K or Lys), and arginine (R or Arg).

The amino acid replacement or substitution can be conservative, semi-conservative, or non-conservative. The phrase “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz and Schirmer, Principles of Protein Structure, Springer-Verlag, New York (1979)). According to such analyses, groups of amino acids may be defined where amino acids within a group exchange preferentially with each other, and therefore resemble each other most in their impact on the overall protein structure (Schulz and Schirmer, supra). Examples of conservative amino acid substitutions include substitutions of amino acids within the sub-groups described above, for example, lysine for arginine and vice versa such that a positive charge may be maintained, glutamic acid for aspartic acid and vice versa such that a negative charge may be maintained, serine for threonine such that a free-OH can be maintained, and glutamine for asparagine such that a free —NH₂ can be maintained. “Semi-conservative mutations” include amino acid substitutions of amino acids within the same groups listed above, but not within the same sub-group. For example, the substitution of aspartic acid for asparagine, or asparagine for lysine, involves amino acids within the same group, but different sub-groups. “Non-conservative mutations” involve amino acid substitutions between different groups, for example, lysine for tryptophan, or phenylalanine for serine, etc.

The bacterium or the delta hemolysin peptide may be engineered such that the bacterium produces and secretes the delta hemolysin peptide outside the bacterium, e.g., in the extracellular environment of the bacterium. Elements for engineering a bacterium to secrete a polypeptide include a signal peptide-encoding sequence placed upstream of, and in-frame with, the coding sequence of the delta hemolysin peptide. A “signal peptide” or “signal peptide sequence” is defined herein as a peptide sequence usually present at the N-terminal end of newly synthesized secretory or membrane proteins which directs the protein across or into a cell membrane of the cell (the plasma membrane in prokaryotes and the endoplasmic reticulum membrane in eukaryotes). The signal peptide is capable of directing the protein into a cellular secretory pathway. It can be subsequently removed following proper localization of the protein. Signal peptides are short peptides, generally 20-30 amino acids in length, and have three distinguishable structural domains with different functions; an amino-terminal region with a net positive charge (the n-region) followed by a hydrophobic region (the h-region) and a protease recognition sequence (the c-region) with a preference for small residues at the −3 (P3) and −1 (P1) positions relative to the cleavage site. The signal peptide may be natural signal peptide, a specific signal peptide to the genus or species of bacteria being engineered, or may be an engineered or non-natural signal peptide. Exemplary signal peptide sequences are available at services.healthtech.dtu.dk/services/SignalP-6.0/. In select embodiments, the signal peptide is a NSP4 signal peptide.

Accordingly, in some embodiments, the delta hemolysin peptide may be linked to a signal peptide. The delta hemolysin peptide may be linked to a signal peptide may be linked via a linker peptide. The linker peptide may be of any sequence or length. In some embodiments, the linker peptide is cleavable. In some embodiments, the linker peptide is non-cleavable.

The bacterium is one that is preferably viable in the gastrointestinal tract of mammals.

In some embodiments, the bacterium is a non-pathogenic bacteria. “Non-pathogenic bacteria” refers to bacteria that are not capable of causing disease or harmful responses in a host. In some embodiments, non-pathogenic bacteria are Gram-negative bacteria. In some embodiments, non-pathogenic bacteria are Gram-positive bacteria. Examples of non-pathogenic bacteria include, but are not limited to Bacillus, Bacteroides, Bifidobacterium, Brevibacteria, Clostridium, Enterococcus, Escherichia, Lactobacillus, Lactococcus, Saccharomyces, and Staphylococcus, e.g., Bacillus coagulans, Bacillus subtilis, Bacteroides fragilis, Bacteroides subtilis, Bacteroides thetaiotaomicron, Bifidobacterium bifidum, Bifidobacterium infantis, Bifidobacterium lactis, Bifidobacterium longum, Clostridium butyricum, Enterococcus faecium, Escherichia coli, Lactobacillus acidophilus, Lactobacillus bulgaricus, Lactobacillus casei, Lactobacillus johnsonii, Lactobacillus paracasei, Lactobacillus plantarum, Lactobacillus reuteri, Lactobacillus rhamnosus, and Lactococcus lactis.

The bacterium may be a commensal bacterium. Commensal bacteria include those species of bacteria that reside on either a surface of the body or at mucosa without harming human health. In some embodiments, commensal bacteria are present in the indigenous microbiota of the gut or gastrointestinal tract.

The bacterium may be a probiotic bacterium. “Probiotic” is used to refer to live, non-pathogenic microorganisms, e.g., bacteria, which can confer health benefits to a host organism (e.g., a mammal (e.g., human)) that contains an appropriate amount of the microorganism. Some species, strains, and/or subtypes of non-pathogenic bacteria are currently recognized as probiotic. Examples of probiotic bacteria include, but are not limited to, Lactobacillus, Bifidobacterium, Streptococcus, Enterococcus, Escherichia, and Bacillus, e.g., Bifidobacterium bifidum, Enterococcus faecium, Escherichia coli, Escherichia coli strain Nissle, Lactobacillus acidophilus, Lactobacillus bulgaricus, Lactobacillus paracasei, and Lactobacillus plantarum. In some embodiments, the bacterium is from the genus Escherichia. In select embodiments, the bacterium is Escherichia coli strain Nissle.

The bacterium may be a variant or a mutant strain of bacterium. For example, non-pathogenic bacteria may be genetically engineered to enhance or improve desired biological properties, e.g., survivability. Non-pathogenic bacteria may also be genetically engineered to provide probiotic properties. Probiotic bacteria may be genetically engineered to enhance or improve probiotic properties. Naturally pathogenic bacteria may be genetically engineered to reduce or eliminate pathogenicity or enhance or improve probiotic properties.

The bacterium may be a food grade bacterium. “Food grade bacteria” refers to any bacteria, alive or dead, that has no harmful effect on human health or that has a GRAS (generally recognized as safe) status. Such bacteria include but are not limited to Bacillus, Carnobacterium, Enterococcus, Lactobacillus, Lactococcus, Leuconostoc, Oenococcus, Pediococcus, Streptococcus, Tetragenococcus, Vagococcus, and Weissella.

The bacterium preferably has a low mutation rate. The bacterium preferably has a mutation rate of less than about 100×10⁻¹⁰ mutations per cell per generation, less than about 60×10⁻¹⁰ mutations per cell per generation, and more preferably less than about 20×10⁻¹⁰ mutations per cell per generation.

In some embodiments, the bacterium is in a subject or is isolated from a subject. For example, the bacterium may be derived from gut bacteria of a subject. Thus, the bacterium may be isolated and engineered ex vivo or the bacterium may be genetically modified in situ in the subject.

Thus, also provided herein are methods of engineering a bacterium. In some embodiments, the methods comprise introducing a nucleic acid encoding a delta hemolysin peptide or variant or polypeptide comprising thereof into the bacterium (e.g., by transfection, transduction, conjugative transfer, lysogenic conversion, and protoplast fusion). In some embodiments, the bacterium is in vitro or ex vivo.

In some embodiments, the bacterium is in a subject. In some embodiments, the methods comprise introducing a nucleic acid encoding a delta hemolysin peptide or variant or polypeptide comprising thereof into the subject with a system engineered to modify the desired bacterium. The nucleic acid encoding a delta hemolysin peptide or variant or polypeptide may be expressed on a conjugative plasmid in a donor bacterium and be transferred into numerous bacterial phyla in a microbiome setting. Embodiments regarding the delta hemolysin peptide and the bacteria described above are equally applicable for these methods.

Further provided are compositions comprising an engineered bacterium, or a population of engineered bacteria, as described herein. The compositions may comprise a population of a single type of engineered bacteria. For example, the composition may comprise a population of engineered Escherichia coli strain Nissle bacteria. Alternatively, the compositions may comprise two or more bacteria populations, each population comprising a different type of engineered bacteria. The two or more populations may comprise two or more types of engineered bacterium as described herein. Alternatively, the two or more populations may comprise at least one population of engineered bacteria as described herein and at least one separate probiotic or engineered bacteria.

The compositions may comprise a pharmaceutically acceptable carrier or excipient as described below. For example, for oral administration, the compositions may include excipients and carriers useful for ingestible tablets, buccal tablets, troches, capsules, elixirs, suspensions, syrups, wafers, and the like. The tablets, troches, pills, capsules, and the like, as described above, may also include a coating (e.g., an enteric coating).

Alternatively or in addition, the bacterium may be incorporated into a food or beverage product, or food or beverage additive. Food and beverage products are any substances that may be used or prepared for use as food or beverage, including, but not limited to, processed or unprocessed food products, meat, meat-containing products, cheese, fruits, vegetables, fruit and vegetable juices, milk and milk products (e.g., dairy, soy, nut), and meal replacement beverages. Food or beverage additives are those compositions engineered to be added to an prepared food or beverage product.

The phrase “pharmaceutically acceptable,” as used in connection with compositions and/or bacteria of the present disclosure, refers to molecular entities and other ingredients of such compositions that are physiologically tolerable and do not typically produce untoward reactions when administered to a subject (e.g., a mammal, a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the nucleic acids, vectors, cells, or therapeutic antibodies) and does not negatively affect the subject to which the composition(s) are administered. Any of the pharmaceutical compositions and/or cells to be used in the present methods can comprise pharmaceutically acceptable carriers, excipients, or stabilizers in the form of lyophilized formations or aqueous solutions.

Pharmaceutically acceptable carriers, including buffers, are well known in the art, and may comprise phosphate, citrate, and other organic acids; antioxidants including ascorbic acid and methionine; preservatives; low molecular weight polypeptides; proteins, such as serum albumin, gelatin, or immunoglobulins; amino acids; hydrophobic polymers; monosaccharides; disaccharides; and other carbohydrates; metal complexes; and/or non-ionic surfactants. See, e.g., Remington: The Science and Practice of Pharmacy 20th Ed. (2000) Lippincott Williams and Wilkins, Ed. K. E. Hoover.

The route by which the disclosed bacteria are administered and the form of the composition will dictate the type of carrier to be used. The composition may be in a variety of forms, suitable, for example, for systemic administration (e.g., oral, rectal, nasal, sublingual, buccal, implants, or parenteral injections) or topical administration (e.g., dermal, pulmonary, nasal, aural, ocular, liposome delivery systems, or iontophoresis). In select embodiments, the disclosed bacteria are administered by oral administration, rectal administration, intravenous injection, nasal administration, and subcutaneous injection.

Carriers for systemic administration typically include at least one of diluents, lubricants, binders, disintegrants, colorants, flavors, sweeteners, antioxidants, preservatives, glidants, solvents, suspending agents, wetting agents, surfactants, cyclodextrins combinations thereof, and others. All carriers are optional in the compositions.

Suitable diluents include sugars such as glucose, lactose, dextrose, and sucrose; diols such as propylene glycol; calcium carbonate; sodium carbonate; sugar alcohols, such as glycerin; mannitol; and sorbitol. Suitable lubricants include silica, talc, stearic acid and its magnesium salts and calcium salts, calcium sulfate; and liquid lubricants such as polyethylene glycol and vegetable oils such as peanut oil, cottonseed oil, sesame oil, olive oil, corn oil and oil of theobroma. Suitable binders include polyvinyl pyrrolidone; magnesium aluminum silicate; starches such as corn starch and potato starch; gelatin; tragacanth; and cellulose and its derivatives, such as sodium carboxymethylcellulose, ethyl cellulose, methylcellulose, microcrystalline cellulose, and sodium carboxymethylcellulose. Suitable disintegrants include agar, alginic acid and the sodium salt thereof, effervescent mixtures, croscarmellose, crospovidone, sodium carboxymethyl starch, sodium starch glycolate, clays, and ion exchange resins. Suitable colorants include a colorant such as an FD&C dye. Suitable flavors include menthol, peppermint, and fruit flavors. Suitable sweeteners include aspartame and saccharin. Suitable antioxidants include butylated hydroxyanisole (“BHA”), butylated hydroxytoluene (“BHT”), and vitamin E. Suitable preservatives include benzalkonium chloride, methyl paraben and sodium benzoate. Suitable glidants include silicon dioxide.

Suitable solvents include water, isotonic saline, ethyl oleate, glycerine, hydroxylated castor oils, alcohols such as ethanol, dimethyl sulfoxide, N-methyl-2-pyrrolidone, dimethylacetamide and phosphate (or other suitable buffer). The amount of solvent(s) in a systemic or topical composition is typically from about 0 to about 100%.

Compositions for oral administration can have various dosage forms. For example, solid forms include tablets, capsules, granules, and bulk powders.

Tablets can be compressed, tablet triturates, enteric-coated, sugar-coated, film-coated, or multiple-compressed. Tablets typically include an active component, and a carrier comprising ingredients selected from diluents, lubricants, binders, disintegrants, colorants, flavors, sweeteners, glidants, and combinations thereof. Specific diluents include calcium carbonate, sodium carbonate, mannitol, lactose, and cellulose. Specific binders include starch, gelatin, and sucrose. Specific disintegrants include alginic acid and croscarmellose. Specific lubricants include magnesium stearate, stearic acid, and talc. Specific colorants are the FD&C dyes, which can be added for appearance. Chewable tablets preferably contain sweeteners such as aspartame and saccharin, or flavors such as menthol, peppermint, fruit flavors, or a combination thereof.

Capsules (including implants, time release and sustained release formulations) typically include a compound as disclosed herein, and a carrier including one or more diluents disclosed above in a capsule comprising gelatin. Granules typically comprise a disclosed compound, and preferably glidants such as silicon dioxide to improve flow characteristics. Implants can be of the biodegradable or the non-biodegradable type.

Solid compositions may be coated by conventional methods, typically with pH or time-dependent coatings, such that a disclosed compound is released in the gastrointestinal tract in the vicinity of the desired application, or at various points and times to extend the desired action. The coatings typically include one or more components selected from the group consisting of cellulose acetate phthalate, polyvinyl acetate phthalate, hydroxypropyl methyl cellulose phthalate, ethyl cellulose, EUDRAGIT® coatings (available from Evonik Industries of Essen, Germany), waxes and shellac.

Compositions for oral administration can have liquid forms. For example, suitable liquid forms include aqueous solutions, emulsions, suspensions, solutions reconstituted from non-effervescent granules, suspensions reconstituted from non-effervescent granules, effervescent preparations reconstituted from effervescent granules, elixirs, tinctures, syrups, and the like. Liquid orally administered compositions typically include a disclosed compound and a carrier, namely, a carrier selected from diluents, colorants, flavors, sweeteners, preservatives, solvents, suspending agents, and surfactants. Peroral liquid compositions preferably include one or more ingredients selected from colorants, flavors, and sweeteners.

Other compositions useful for attaining systemic delivery of the subject compounds include sublingual, buccal and nasal dosage forms. Such compositions typically include one or more of soluble filler substances such as diluents including sucrose, sorbitol, and mannitol; and binders such as acacia, microcrystalline cellulose, carboxymethyl cellulose, and hydroxypropyl methylcellulose. Such compositions may further include lubricants, colorants, flavors, sweeteners, antioxidants, and glidants.

### Nucleic Acids

The present disclosure also provides for nucleic acids encoding delta hemolysin peptide or variant or polypeptide comprising thereof and vectors containing or encoding these nucleic acids. The vectors may be used to propagate the nucleic acid in an appropriate cell and/or to allow expression from the nucleic acid (e.g., an expression vector). The person of ordinary skill in the art would be aware of the various vectors available for propagation and expression of a nucleic acid sequence.

The present disclosure further provides engineered, non-naturally occurring vectors and vector systems, which can encode delta hemolysin peptide or variant or polypeptide comprising thereof. The vector(s) can be introduced into a cell that is capable of expressing protein or peptide encoded thereby, including any suitable prokaryotic cell.

In one embodiment, a nucleic acid encoding the disclosed delta hemolysin peptide or variant or polypeptide comprising thereof is contained in a plasmid vector that allows expression of the disclosed delta hemolysin peptide or variant or polypeptide comprising thereof and subsequent isolation and purification of from the recombinant vector. Accordingly, the disclosed delta hemolysin peptide or variant or polypeptide comprising thereof disclosed herein can be purified following expression, obtained by chemical synthesis, or obtained by recombinant methods.

To construct cells that express the disclosed delta hemolysin peptide or variant or polypeptide comprising thereof, expression vectors for stable or transient expression of the disclosed delta hemolysin peptide or variant or polypeptide comprising thereof may be constructed via conventional methods as described herein and introduced into host cells. For example, nucleic acids encoding the disclosed delta hemolysin peptide or variant or polypeptide comprising thereof may be cloned into a suitable expression vector, such as a plasmid or a viral vector in operable linkage to a suitable promoter.

In certain embodiments, vectors of the present disclosure can drive the expression of one or more sequences in prokaryotic cells. Promoters that may be used include T7 RNA polymerase promoters, constitutive E. coli promoters, and promoters that could be broadly recognized by transcriptional machinery in a wide range of bacterial organisms. As described above, various bacterial hosts can be engineered to produce the delta hemolysin peptide or variant or polypeptide comprising thereof disclosed herein.

Additionally, the vector may contain, for example, some or all of the following: a selectable marker gene, such as the neomycin gene for selection of stable or transient transfectants in host cells; enhancer/promoter sequences from the immediate early gene of human CMV for high levels of transcription; transcription termination and RNA processing signals from SV40 for mRNA stability; 5′- and 3′-untranslated regions for mRNA stability and translation efficiency from highly-expressed genes like α-globin or β-globin; SV40 polyoma origins of replication and ColE1 for proper episomal replication; internal ribosome binding sites (IRESes), versatile multiple cloning sites; T7 and SP6 RNA promoters for in vitro transcription of sense and antisense RNA; a “suicide switch” or “suicide gene” which when triggered causes cells carrying the vector to die (e.g., HSV thymidine kinase, an inducible caspase such as iCasp9), and reporter gene for assessing expression of the chimeric receptor. Suitable vectors and methods for producing vectors containing transgenes are well known and available in the art. Selectable markers also include chloramphenicol resistance, tetracycline resistance, spectinomycin resistance, streptomycin resistance, erythromycin resistance, rifampicin resistance, bleomycin resistance, thermally adapted kanamycin resistance, gentamycin resistance, hygromycin resistance, trimethoprim resistance, dihydrofolate reductase (DHFR), GPT; the URA3, HIS4, LEU2, and TRP1 genes of S. cerevisiae.

When introduced into the cell, the vectors may be maintained as an autonomously replicating sequence or extrachromosomal element or may be integrated into host DNA.

Vectors according to the present disclosure can be transformed, transfected, or otherwise introduced into cells. Transfection refers to the taking up of a vector by a cell whether or not any coding sequences are in fact expressed. Numerous methods of transfection are known to the ordinarily skilled artisan, for example, lipofectamine, calcium phosphate co-precipitation, electroporation, DEAE-dextran treatment, microinjection, viral infection, and other methods known in the art.

### Methods

Further disclosed herein are methods for improving or maintaining gut health, regulating glucose metabolism, maintaining intestinal integrity, and/or promoting tissue repair and regeneration (e.g., gastrointestinal tissue repair and regeneration).

The methods comprise modulating GLP-1 activation in the gut.

In some embodiments, the methods comprise introducing to a subject in need thereof an effective amount of a bacterium as described herein, or a population or composition comprising thereof. The engineered bacteria may be administered with a pharmaceutically acceptable carrier or excipient as a composition, described above.

In some embodiments, the methods may comprise administering to the subject in vitro or ex vivo engineered bacteria. Alternatively, the methods may comprise engineering bacteria in vivo. Thus, the methods may comprise introducing a nucleic acid encoding a delta hemolysin peptide or variant or polypeptide comprising thereof into the subject with a system engineered to modify the desired bacterium, e.g., CRISPR-Cas systems, transposon mutagenesis, conjugate systems. In some embodiments, the nucleic acid encoding a delta hemolysin peptide or variant or polypeptide may be expressed on a conjugative plasmid in a donor bacterium and be transferred into numerous bacterial phyla in a microbiome setting.

In some embodiments, the methods may comprise administering to the subject a GLP-1 receptor agonist. GLP-1 receptor agonists are a class of medications that mimic the action of the glucagon-like peptide-1 (GLP-1), a hormone involved in glucose metabolism. GLP-1 receptor agonists include exenatide (exendin-4), liraglutide, dulaglutide, semaglutide, lixisenatide, GLP-1 variants, and analogues and derivatives thereof.

In some embodiments, the methods comprise administering to a subject in need thereof a delta hemolysin peptide or variant or polypeptide comprising thereof, as described above, compositions comprising thereof, or a nucleic acid encoding thereof.

The delta hemolysin peptide may be administered with a pharmaceutically acceptable carrier or excipient as a composition, described above. In addition to the carriers and excipients described above the delta hemolysin peptide may be delivered using a delivery vehicle such as nanoparticle- and lipid-based protein delivery systems. For example, the delta hemolysin peptide may be in a liposome in which peptide is combined with amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

Alternatively, a nucleic acid encoding the delta hemolysin peptide configured for expression may be delivered to the subject. For example, a vector(s) capable of expressing the delta hemolysin peptide (e.g., a mammalian expression vector) can be introduced into a cell in a subject. Viral and non-viral based gene transfer methods can be used to introduce nucleic acids encoding components of the present system into cells, tissues, or a subject. Non-viral vector delivery systems include DNA plasmids, cosmids, RNA (e.g., a transcript of a vector described herein), a nucleic acid, and a nucleic acid complexed with a delivery vehicle. Viral vector delivery systems include DNA and RNA viruses, which have either episomal or integrated genomes after delivery to the cell. Viral vectors include, for example, retroviral, lentiviral, adenoviral, adeno-associated and herpes simplex viral vectors. Vectors can comprise any of a number of promoters known to the art, wherein the promoter is constitutive, regulatable or inducible, cell type specific, tissue-specific, or species specific.

In some embodiments, in the methods disclosed herein, the subject has or is suspected of having a disease or disorder. In some embodiments, the disease or disorder comprises a gastrointestinal disease or disorder and/or an inflammatory disease or disorder, including diseases and disorders that have an inflammatory component or response.

The methods may also be used for treating a disease or disorder.

In select embodiments, the subject has or is suspected of having an inflammatory disease or disorder. Inflammatory diseases are characterized by activation of the immune system in a tissue or an organ to abnormal levels that may lead to abnormal function and/or disease in the tissue or organ.

In select embodiments, the subject has or is suspected of having a gastrointestinal disease or disorder. Gastrointestinal disease and disorders include a wide range of diseases affecting the esophagus, liver, stomach, small and large intestines, gallbladder, and pancreas. Exemplary gastrointestinal diseases and disorders include, but are not limited to, irritable bowel syndrome (IBS), colitis (e.g., infectious colitis, ulcerative colitis, Crohn's disease, ischemic colitis, radiation colitis), colon polyps and cancer, peptic ulcer disease, gastritis, gastroenteritis, celiac disease, gallstones, fecal incontinence, lactose intolerance, Hirschsprung disease, abdominal adhesions, Barrett's esophagus, appendicitis, indigestion (dyspepsia), intestinal pseudo-obstruction, pancreatitis, short bowel syndrome, Whipple's disease, Zollinger-Ellison syndrome, malabsorption syndromes and hepatitis. In some embodiments, the subject has or is suspected of having an inflammatory bowel disease. In some embodiments, the subject has or is suspected of having Crohn's disease. In some embodiments, the subject has or is suspected of having ulcerative colitis.

In some embodiments, an effective amount of engineered bacteria, composition, or population comprising thereof, a delta hemolysin peptide, a composition comprising a delta hemolysin peptide, or a nucleic acid encoding a delta hemolysin peptide as described herein can be administered. As used herein the term “effective amount” may be used interchangeably with the term “therapeutically effective amount” and refers to that quantity that is sufficient to result in a desired activity upon administration to a subject in need thereof. Within the context of the present disclosure, the term “effective amount” refers to that quantity to improve or maintain gut health, regulate glucose metabolism, maintain intestinal integrity, promote tissue repair and regeneration, and/or treat or prevent a disease or disorder.

When utilized as a method of treatment, the effective amount may depend on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. In some embodiments, the effective amount alleviates, relieves, ameliorates, improves, reduces the symptoms, or delays the progression of any disease or disorder in the subject. In some embodiments, the subject is a human.

In the context of the present disclosure insofar as it relates to any of the disease conditions recited herein, the terms “treat,” “treatment,” and the like mean to relieve or alleviate at least one symptom associated with such condition, or to slow or reverse the progression of such condition. Within the meaning of the present disclosure, the term “treat” also denotes to arrest, delay the onset (e.g., the period prior to clinical manifestation of a disease) and/or reduce the risk of developing or worsening a disease.

A wide range of second therapies may be used in conjunction with the methods of the present disclosure. The second therapy may be administered at the same time as the initial therapy, either in the same composition or in a separate composition administered at substantially the same time as the first composition. In some embodiments, the second therapy may precede or follow the treatment of the first therapy by time intervals ranging from hours to months. The second therapy may be administration of an additional therapeutic agent or may be a second therapy not connected to administration of another agent.

The additional therapeutic agent may comprise immunosuppressants (e.g., azathioprine, mercaptopurine, cyclosporine, tacrolimus, methotrexate, mycophenolate mofetil, rapamycin, sirolimus), anti-inflammatory agents (e.g., corticosteroids and aminosalicylates), immunotherapies, antibiotics, and analgesics.

The additional therapeutic agent(s) may be administered to a subject by a variety of methods. In any of the uses or methods described herein, administration may be by various routes known to those skilled in the art, including without limitation oral, inhalation, intravenous, intramuscular, topical, subcutaneous, systemic, and/or intraperitoneal administration to a subject in need thereof. The additional therapeutic agent may be administered by parenteral administration (including, but not limited to, subcutaneous, intramuscular, intravenous, intraperitoneal, intracardiac and intraarticular injections). In some embodiments, the additional therapeutic agent may be administered in the same or different manner than the disclosed engineered bacteria, compositions or populations comprising thereof, delta hemolysin peptides, compositions comprising a delta hemolysin peptide, or nucleic acids encoding a delta hemolysin peptide.

### Systems or Kits

Also provided herein are systems or kits for carrying out the disclosed methods. In some embodiments, the kit comprises engineered bacteria. In some embodiments, the kit comprises a population of bacteria and the nucleic acid or vector thereof encoding a delta hemolysin peptide or variant or polypeptide comprising thereof. The kit may further comprise reagents for engineering a bacterium including for example, transfection or transformation reagents, vectors or plasmids, donor bacteria, and the like.

The kit may be supplied in a solid (e.g., lyophilized) or liquid form. The various components of the kit of the present disclosure may optionally be contained within different containers (e.g., vial, ampoule, test tube, flask, or bottle) for each individual component (e.g., amplification oligonucleotides, probe oligonucleotides, or buffer). Each component will generally be suitable as aliquoted in its respective container or provided in a concentrated form. Other containers suitable for conducting certain steps of the amplification/detection assay may also be provided. The individual containers are preferably maintained in close confinement for commercial sale.

The kit may include instructions for use in any of the methods described herein. The instructions can comprise a description of administration to a subject to achieve the intended effect. The instructions generally include information as to dosage, dosing schedule, and route of administration for the intended treatment.

The kits provided herein are in suitable packaging. Suitable packaging includes, but is not limited to, vials, bottles, jars, flexible packaging, and the like. A kit may have a sterile access port (for example, the container may be an intravenous solution bag or a vial having a stopper pierceable by a hypodermic injection needle). The container may also have a sterile access port.

The packaging may be unit doses, bulk packages (e.g., multi-dose packages) or sub-unit doses. Instructions supplied in the kits of the disclosure are typically written instructions on a label or package insert. The label or package insert indicates that the pharmaceutical compositions are used for treating, delaying the onset, and/or alleviating a disease or disorder in a subject.

### EXAMPLES

The following are examples of the present invention and are not to be construed as limiting.

### Materials and Methods

Mice and diets C57BL/6J and Il10^{-/-} mice were purchased from Jackson Laboratory. Animals were purchased from maximum barrier rooms and maintained in high barrier animal rooms at Columbia University. All mice were maintained under specific-pathogen-free (SPF) conditions for the duration of the experiments. All experiments were performed under IACUC approved guidelines. To control for microbiota and cage effects, mice were shuffled between cages before the start of each experiment and randomly assigned to each group. All experiments were performed with gender and age (8-12 weeks at start of experiment) matched mice. Animals were maintained on normal diet (PicoLab Rodent Diet 20, 5053) until start of experiments and then switched to fiber-deficient diet (Teklad Custom Diet, TD.00278) or continued on ND as indicated. ND contained 21% protein, 53.4% carbohydrates, 5% fat, 5% fiber and 4 kcal/g. FDD contained 19.3% protein, 64.8% carbohydrates, 5.1% fat and no fiber, overall the diet provides 3.9 kcal/g.

DSS colitis and GLP-1 modulating therapies For induction of acute DSS colitis, between 0.5% to 6% w/v dextran sulfate sodium (DSS, colitis grade, MPbio) was supplemented in drinking water for five days. Drinking water was furthermore enriched with 2% w/v sucrose for all groups. Mice were switched to FDD seven days prior to DSS supplementation or maintained on ND during course of experiment. As indicated, FDD mice were i.p. injected with 0.1 μg/g GLP-1 receptor agonist (Exendin-4, AS-24463, AnaSpec) or 0.4 μg/g GLP-1 receptor antagonist (Exendin-9-39, AS-24467, AnaSpec) in 100 μl PBS every day starting one day before start of DSS. Additionally, mice were orally gavaged with either 10¹⁰ CFU E. coli Nissle (EcN) or 10¹⁰ CFU E. coli Nissle-Hld_{SE} (EcN-Hld_{SE}) every day starting one day prior to DSS supplementation. Of note, tissue samples for RNA-sequencing analysis are derived from mice either gavaged with EcN (control and GLP-1 receptor agonist groups) or EcN-Hld_{SE} (EcN-Hld_{SE} and GLP-1 receptor antagonist groups). Mice were monitored daily for changes in body weight. After five days, mice were euthanized for blood and tissue sampling.

Lamina propria cell isolation and flow cytometry analysis Lymphocyte isolation from colon was performed as previously described (Parigi, S. M., et al., Sci Rep, 2018. 8 (1): p. 154). In brief, large intestine was removed and opened longitudinally. After washing of tissue in PBS, the intestine was cut into 0.5 cm long pieces and incubated in 5 mM EDTA solution (RPMI, 5% FCS) twice for 20 min at 37° C. under agitation of 600 rpm. After washing with PBS, lamina propria cells were isolated by digesting the tissue pieces with Collagenase D, DNAse and Dispase (in RPMI, 10% FCS) for 50 min at 37° C. under agitation of 1000 rpm. The supernatant of the digest was pelleted and lymphocytes were further purified using 80:40 Percoll gradient. After isolation, cells were analyzed immediately by flow cytometry. For surface staining of fresh cells, cell suspensions were blocked with Fc-blocking solution and stained with antibody mix for 20 min at 4° C. Data acquisition was performed using a 5-laser Fortessa flow cytometer (BD Bioscience) and analysis was carried out using FlowJo software (TreeStar).

Histological analyses The colonic tissue was carefully rinsed with PBS to remove fecal matter. Colon and small intestine was fixated in 10% formalin for >24 hours and embedded in paraffin. 5 μm section were stained with H&E, Alcian Blue and Immunohistochemistry (IHC) staining for GLP-1 (GLP-1 polyclonal antibody, PA5-79303, Invitrogen). A pathologist assessed and scored H&E stained slides for colitis grading.

Plating for liver colony forming units (CFU) For bacterial extraction from liver tissue, livers were first weighed and then transferred into sterile stainless steel microvials (Biospec #2007) containing two stainless steel beads (6.35 mm, Biospec #11079635SS) and capped with silicone rubber caps (Biospec #2008). The vials were subsequently filled with 500 μL of sterile phosphate-buffered saline (PBS), and the tubes were placed on ice to prevent bacterial growth. The samples were homogenized using a Mini-BeadBeater-16 (BioSpec) with three cycles of 30 seconds at 4° C. After extraction, the samples were diluted as needed in PBS, and 100 μL of the diluted solution was plated onto Luria-Bertani (LB) agar plates (2% agar). The plates were thoroughly dried and incubated at 37° C. overnight. Colony-forming units (CFU) were counted the following day and normalized to tissue weight.

Plasma analyses GLP-1 and pro-inflammatory cytokine measurements were performed from plasma samples. After euthanasians, blood was drawn from mice with needles coated with EDTA to stop coagulation. Blood samples were pelleted by centrifugation for 10 min at 15000 rpm at 4° C. Supernatant was transferred to a new tube, snap frozen and stored at −80° C. GLP-1 ELISA (GLP-1 Multispecies ELISA kit, BMS2194, Invitrogen) and LEGENDplex Mouse Inflammation Panel (740446, BioLegend) were performed according to manufacturer protocol.

Quantitative PCR 3 mm of distal colon was transferred into 2 ml Lysin Matrix D tubes (6913050, MP Biomedicals) containing 1 ml TRIzol reagent (15596026, Invitrogen), and tissue was disrupted by bead beating. RNA was isolated according to manufacturer's procedures. RNA was subjected to reverse transcription with QScript cDNA SuperMix (QuantaBio). Q-PCR was performed using SYBER Green. Samples were analyzed using the DDCt method and normalized to Gapdh.

RNA colonic transcriptomics For mRNA sequencing using the Illumina platform, total RNA was first extracted from tissue using TRItol reagent. The extracted RNA was quantified using Bioanalyzer to ensure high-quality RNA (RIN>7) for further library preparation. Poly-A mRNA was isolated using the Dynabeads™ mRNA Purification Kit (Thermo Fisher, #61006), followed by fragmentation with the NEBNext Ultra II RNA Library Prep Kit (New England Biolabs, #E7770) at 94° C. for 8 minutes. First-strand cDNA synthesis was performed using SuperScript IV Reverse Transcriptase (Thermo Fisher, #18090050), followed by second-strand cDNA synthesis. After adapter ligation with Illumina TruSeq Adapters (Illumina, #20015960), the cDNA libraries were purified using AMPure XP Beads (Beckman Coulter, #A63881), and amplified using NEBNext High-Fidelity PCR Master Mix (NEB, #M0541). Libraries were validated for quality and size distribution using 2% agarose gel and enriched using size selection beads to eliminate any cDNA below 350 bp, and quantified using a Qubit 4 Fluorometer (Thermo Fisher, #Q33226). Sequencing was performed on an Illumina NextSeq550 platform using paired-end 150 bp reads high output.

Post-sequencing, FastQC (v0.11.9) was employed to assess raw read quality, evaluating metrics such as per-base sequence quality (Q30 or higher), GC content, and potential adapter contamination. Any necessary trimming of low-quality sequences and adapters was carried out using Trim Galore. Reads alignment and transcripts quantification of gene expression were done using Kallisto (v0.46.1) and DESeq2 (v1.30.1) was then used for differential expression analysis, incorporating normalization and variance-stabilizing transformation to account for differences in sequencing depth and library size. Biological replicate consistency was checked using Principal Component Analysis (PCA) to ensure reliable clustering of replicates, and any outliers were flagged for further examination or removal. Functional enrichment analysis was conducted using Gene Set Enrichment Analysis (GSEA) and KEGG pathway enrichment analysis. GSEA was performed using GSEA v4.3.2 software (Broad Institute, Inc., Massachusetts Institute of Technology, and Regents of the University of California). To further isolate the core changes due to GLP-1 modulating therapies, a ranked list of genes was generated based on the average log 2 fold changes of following comparisons of colon transcriptome in DSS colitis of FDD-fed mice: control vs. GLP-1 receptor agonist, control vs. EcN-Hld_{SE}, EcN-Hld_{SE}+GLP-1 receptor antagonist vs. EcN-Hld_{SE}. This ranked list of genes was used to run GSEAP reranked.

16S sequencing Genomic DNA was extracted from bacterial samples using a bead-beating protocol, followed by purification with SPRI beads. The V4 region of the 16S rRNA gene was amplified using modified EMP primers (505f and 806rB), which included Illumina adapters and dual indices for sample identification. PCR products were confirmed via agarose gel electrophoresis (~390 bp), and amplicons were pooled in equimolar concentrations based on qPCR results. After purification using a 2×SPRI bead cleanup and gel extraction, the pooled libraries were sequenced on the Illumina MiSeq platform with paired-end 150 bp reads, using a 20% PhiX spike-in to ensure sequencing quality.

Raw sequencing data were processed using the UPARSE pipeline in USEARCH (v10). Quality filtering removed reads with expected errors above 1.0, and paired-end reads were merged. Primers were trimmed, and sequences were dereplicated before clustering into operational taxonomic units (OTUs) at 97% similarity using the UPARSE-OTU algorithm, which included chimera removal. Amplicon sequence variants (ASVs) were generated using the unoise3 algorithm for higher-resolution sequence identification. Taxonomic classification was performed using a naive Bayes classifier trained on the SILVA database (v138), assigning taxonomy to the genus level where possible.

The ASV table was normalized using total sum scaling (TSS) to account for varying sequencing depth across samples. Statistical comparisons of community composition were made using permutational multivariate analysis of variance (PERMANOVA) with 999 permutations. Differential abundance analysis was conducted using DESeq2 to identify taxa significantly associated with experimental conditions, and beta diversity analysis was performed using UniFrac distances followed by principal coordinate analysis (PCoA) to visualize sample clustering based on microbial community composition between samples.

Engineering EcN-Hld_{SE} E. coli Nissle was used as a bacterial chassis to develop a probiotic strain that secretes the Hld_{SE} peptide from S. epidermidis in vivo. Two different signal peptides were explored: PelB_{SS}, commonly used for heterologous protein secretion in E. coli, and the NSP4 peptide, which has recently been shown to improve secretion efficiency and outperform PelB_{SS}. To avoid potential toxicity from the delta-toxin peptide, the construct was cloned into the pCOLADuet™-1 vector (Novagen #71406), a medium-copy number plasmid optimized for protein expression. Two gBlocks (IDT) were synthesized, each containing a synthetic strong promoter (J23100, Table 1, bold text), a signal peptide (Table 1, underlined text), and the Hld_{SE} sequence (Table 1, italic text). These constructs were cloned into the pCOLADuet™-1 vector using the forward primer: CTTACATTAATTGCGTTGCGTTGACGGCTAGCTCAGTC (SEQ ID NO: 2) and the reverse primer: GATACCGGTAGGCTGCATTTTTTTAAATTTGTTCACGGTATCAAT (SEQ ID NO: 3). The plasmids were assembled using NEBuilder HiFi DNA Assembly Master Mix (NEB #E2621) and transformed into E. coli Nissle, with transformants selected on kanamycin-containing media. All constructs were sequenced verified

TABLE 1

| | | |
|---|---|---|
| pCOLA_NSP4_ | TTGACGGCTAGCTCAGTCCTAGGTAC | SEQ ID |
| Hld_{SE} | AGTGCTAGCTACTAGAGAAAGAGGAG | NO: 4 |
| | AAATACTGATGAAAAAGATTACCGCT | |
| | GCTGCTGGTCTGCTGCTCCTCGCTGC | |
| | CCAGCCGGCGATGGCGATGGCGGCGG | |
| | ATATTATTAGCACCATTGGCGATCTG | |
| | GTGAAATGGATTATTGATACCGTGAA | |
| | CAAATTTAAAAAA | |
| pCOLA_PELB5_ | TTGACGGCTAGCTCAGTCCTAGGTAC | SEQ ID |
| Hld_{SE} | AGTGCTAGCTACTAGAGAAAGAGGAG | NO: 5 |
| | AAATACTAGATGAAGTACCTGCTGCC | |
| | GACCGCGGCGGCGGGTCTGCTGCTGC | |
| | TGGCGGCGCAGCCGGCGATGGCGGAC | |
| | GATGACGATGACATGGCGGCGGATAT | |
| | TATTAGCACCATTGGCGATCTGGTGA | |
| | AATGGATTATTGATACCGTGAACAAA | |
| | TTTAAAAAA | |

### Example 1

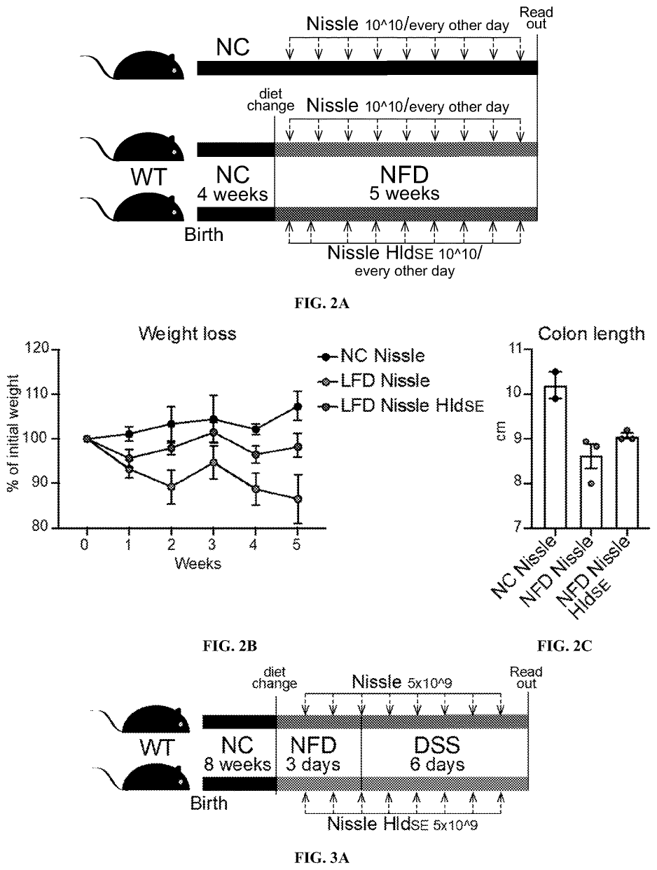

Following 4 weeks of a normal diet, mice were transitioned to a diet low in fiber, known to cause gut barrier dysfunction (FIG. 2A). Treatment with engineered Nissle bacteria producing the delta-hemolysin peptide (Nissle Hld_{SE}) improved clinical signs such as weight loss, (FIG. 2B) which can indicate systemic effects of colitis or barrier dysfunction in the gut, and colon shortening (FIG. 2C), indicative of epithelial damage.

A diet low in fiber also increases acute colitis susceptibility. Herein, a mouse model of colitis was employed dextran sodium sulfate (DSS), a chemical colitogen with anticoagulant properties, to induce disease. DSS is a water-soluble, negatively charged sulfated polysaccharide with a highly variable molecular weight ranging from 5 to 1400 kDa. The mechanism by which DSS induces intestinal inflammation is unclear but is likely the result of damage to the epithelial monolayer lining the large intestine allowing the dissemination of proinflammatory intestinal contents (e.g., bacteria and their products) into underlying tissue.

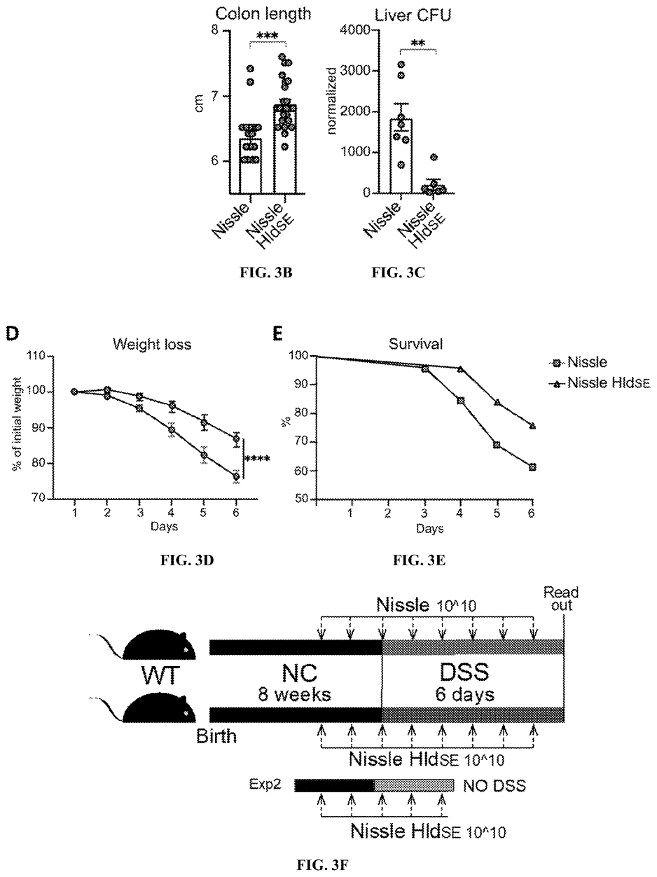

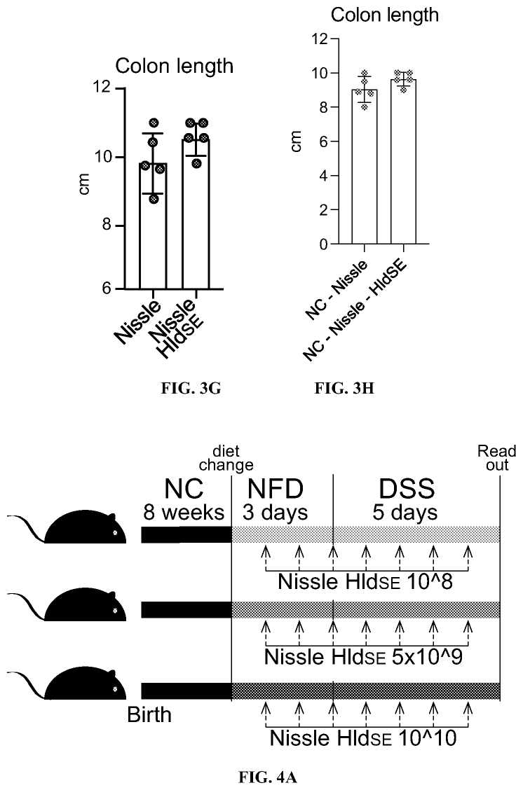

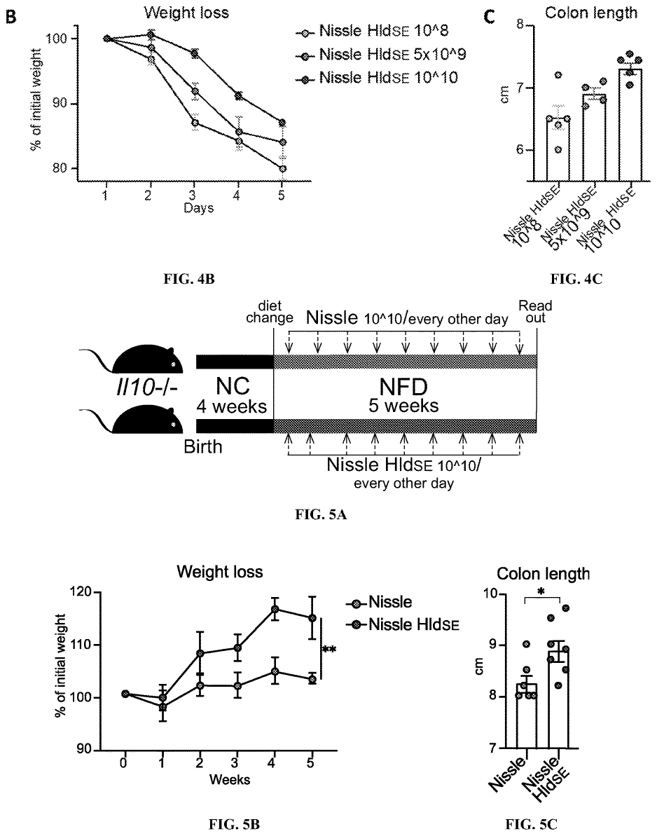

Here, mice with low fiber diets were treated with DSS for 6 days with or without the engineered Nissle Hld_{SE} (FIG. 3A) Treatment with Nissle Hld_{SE} improved barrier function measured based on infiltrating gut bacteria in liver (Liver CFU; FIG. 3C). In healthy mice liver is free of any bacteria but when barrier function is disrupted, gut bacteria can infiltrate into liver. Other clinical signs such as colon length (FIG. 3B), weight loss (FIG. 3D) and survival (FIG. 3E) were also improved. Even without ‘leaky gut’ induced by NFD, mice showed improvement in colon length in acute colitis model under normal chow conditions (FIGS. 3F-3G). Mice also showed a trend in increase of colon length when they were not treated with DSS and fed normal chow (FIG. 3H). The positive effects of Nissle Hld_{SE} treatment of acute colitis are dose dependent over two orders of magnitude (1×10\hat{ }8 to 1×10\hat{ }10 CFU/mouse; FIGS. 4A-4C).

Similar experiments as those described above were carried out in Il10^{-/-} mice (FIG. 5A). Il10^{-/-} mice develop spontaneous, chronic colitis due to inflammatory defect, which is escalated by NFD. Treatment with Nissle Hld_{SE} improved weight loss (FIG. 5B) and colon length (FIG. 5C) in chronic colitis.

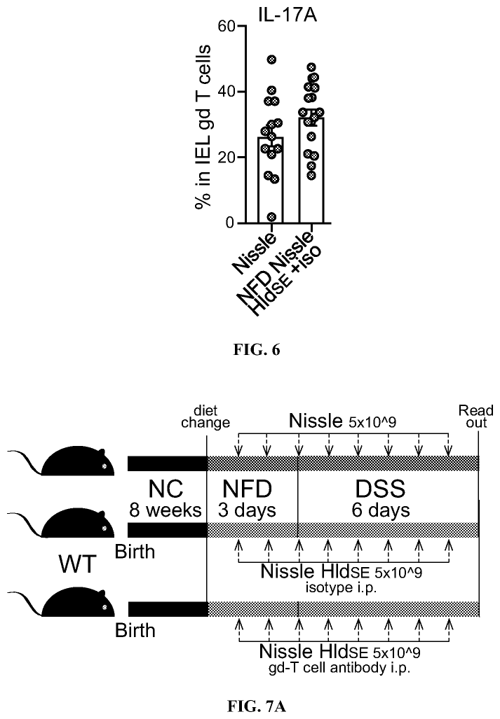

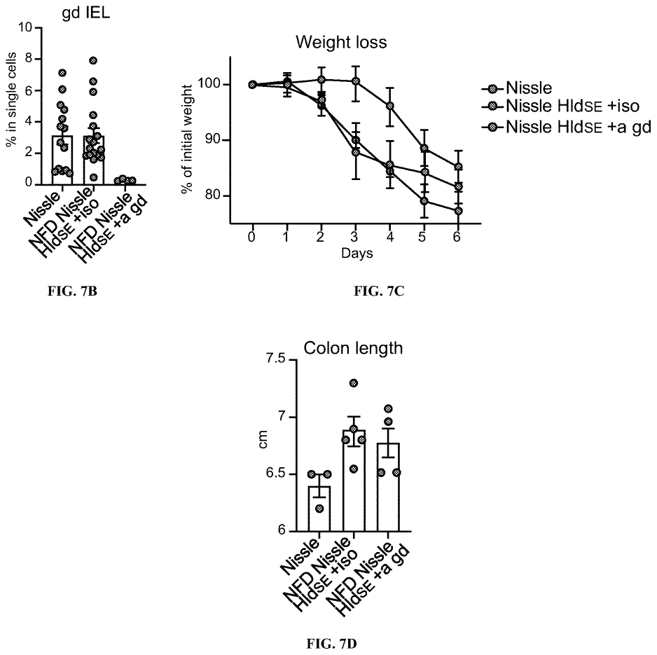

As shown in FIG. 6, an upregulation of IL-17A production by γδ T cells was observed upon introducing the Hld_{SE} peptide in the mouse, highlighting a potential mechanism for improved intestinal integrity and function through the promotion of wound healing by γδ (gd) T cells. An experimental depletion of γδ T cells suggested a notable, albeit not exhaustive, diminishment in the efficacy of the peptide (FIGS. 7A-7D). Upon treatment with Nissle Hld_{SE} intraepithelial γδ T lymphocytes produce more tissue protective IL-17A. When γδ T lymphocytes are depleted, Nissle Hld_{SE} does not prevent rapid weight loss during acute colitis.

### Example 2

### Fiber Deficiency Disrupts Immune, Metabolic and Endocrine Homeostasis in the Gut

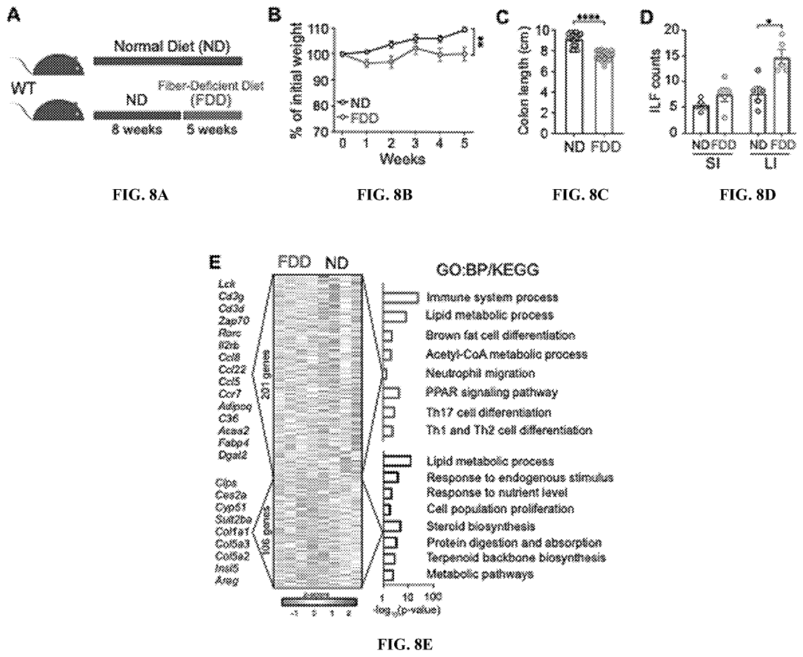

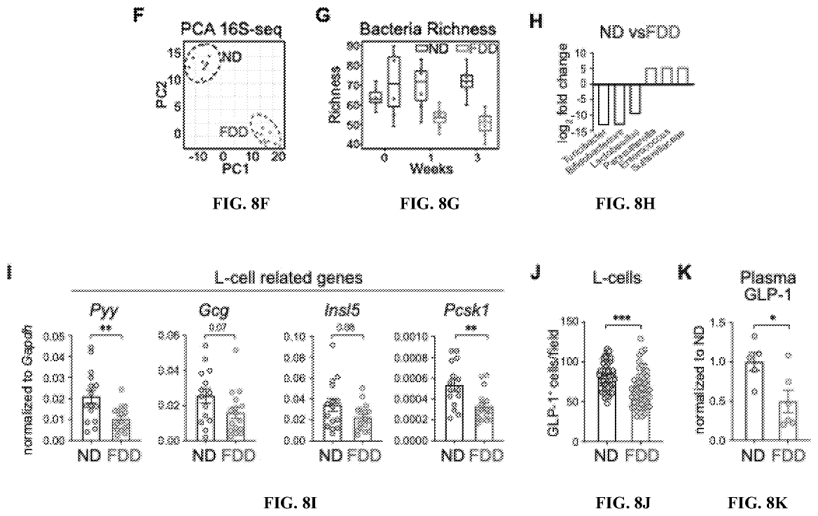

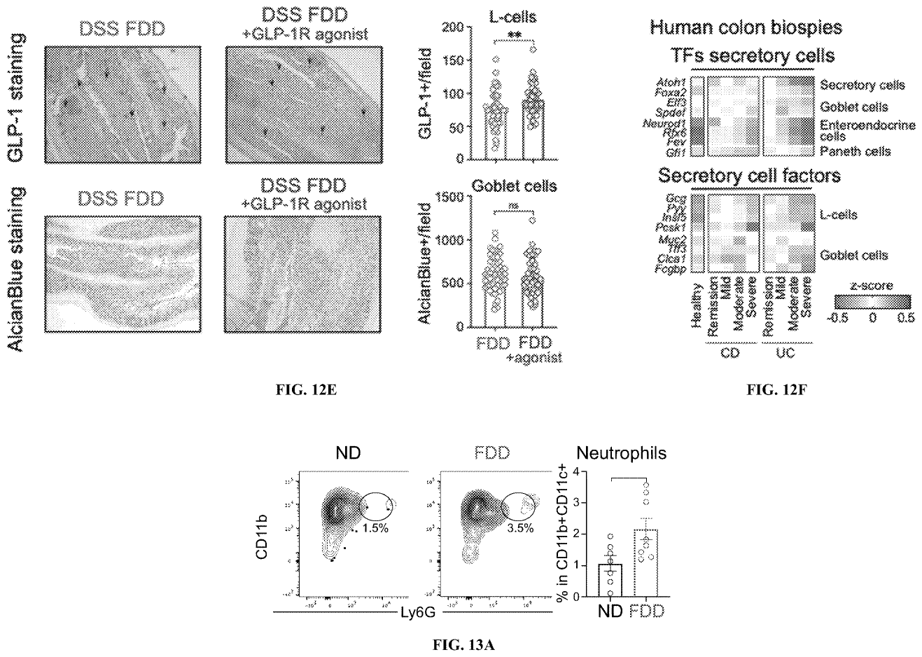

To investigate the impact of dietary fiber on gut health, specific pathogen-free (SPF) mice fed a normal diet (ND) were compared to those fed a fiber-deficient diet (FDD) for five weeks (FIG. 8A). FDD and ND had comparable caloric content per gram of food and similar nutrient profiles. However, FDD contained a higher proportion of carbohydrates in place of fiber (see Methods). Reduced weight gain over time and a shortening of the colon was observed, indicating structural changes in the intestinal tissue (FIGS. 8B and 8C). Histological analysis revealed an increase in isolated lymphoid follicles (ILF) specifically in the large intestine (FIG. 8D), but no overt inflammation. Consistently, over-representation analysis of differentially expressed genes (DEGs) in the colon tissue of FDD mice revealed upregulation of genes involved in immune processes and T cell activation (e.g., Lck, Cd3, Zap70, Rorc, Il2rb), neutrophil migration (e.g., Ccl8, Ccl22, Ccl5, Ccr7), and lipid metabolism (e.g., Cd36, Dgat2, Fabp4, Acca2), including upregulation of Cd36, which regulates lipid and cholesterol uptake (FIG. 8E). In contrast, ND mice showed enrichment in pathways related to different lipid metabolic pathways such as aldosterone biosynthesis (e.g., Dab2, Wnt4) or prostaglandin and eicosanoid secretion (e.g., Pla2g3, Ptges, Pcsk9), as well as extracellular matrix organization (e.g., Col3a1, Col5a3), and sterol and cholesterol biosynthesis (e.g., Sult2ab, Cyp51) (FIG. 8E). Neutrophils in the lamina propria (LP) were elevated, which confirmed the increased immune cell activation in the colon of FDD-fed mice (FIG. 13A). Coupled with the downregulation of genes encoding apical junction proteins (Ocln, Cldn8, Marveld3) (FIG. 13B), these findings suggested weakened barrier function under fiber-deficient conditions. Concurrently, FDD resulted in a significant reduction in bacterial richness, accompanied by a decrease in fiber-dependent strains such as Duncaniella, Lactobacillus and Paramuribaculum, including many SCFA producing strains, and a relative increase of Akkermansia (FIGS. 8F-8H).

A trend towards reduced gene expression of L-cell-derived peptide hormones (Pyy, Insl5, Gcg), and the proglucan processing enzyme Pcsk1 (encoding prohormone convertase 1/3) was observed in the colonic tissue of FDD-fed mice (FIG. 8I). This was accompanied by a reduced number of L-cells, as indicated by decreased GLP-1-positive cells in the colon (FIG. 8J, 13C). Enzyme-immunoassay (EIA) of plasma samples revealed reduced levels of circulating GLP-1 in mice fed FDD for one week (FIG. 8K). FDD results in dysbiosis and disrupts immune, metabolic, and endocrine homeostasis in the gut.

### Example 3

### Release of GLP-1 in Response to Tissue Damage is Stunted Under Fiber Deficient Condition

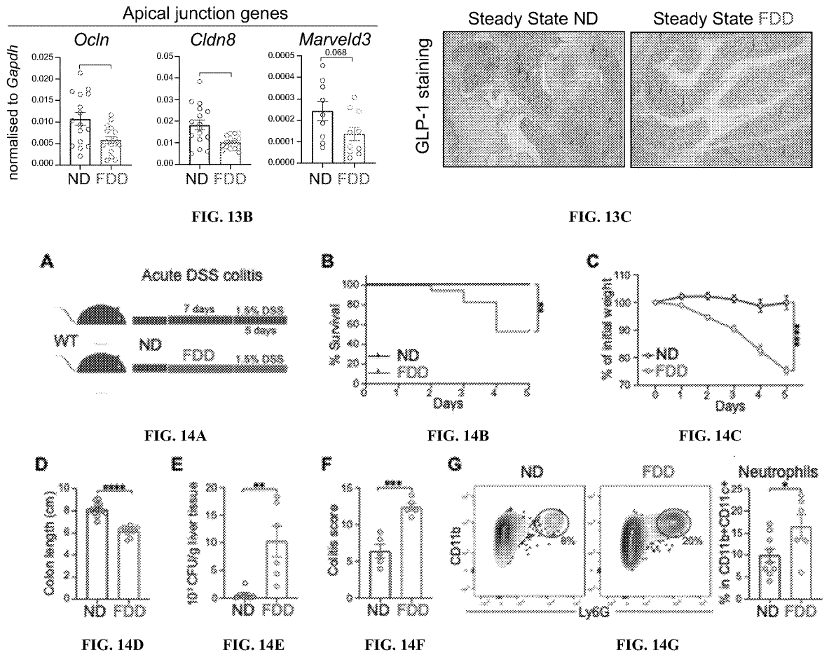

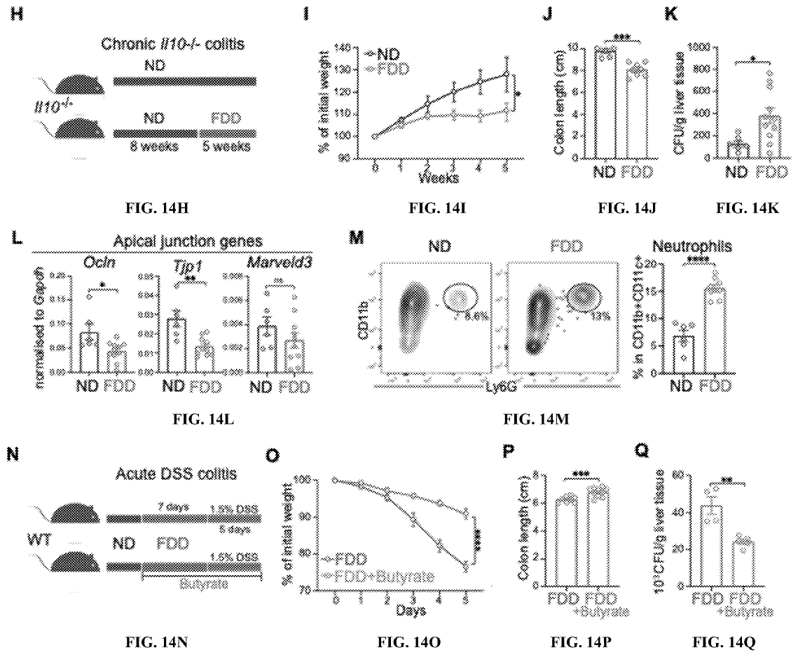

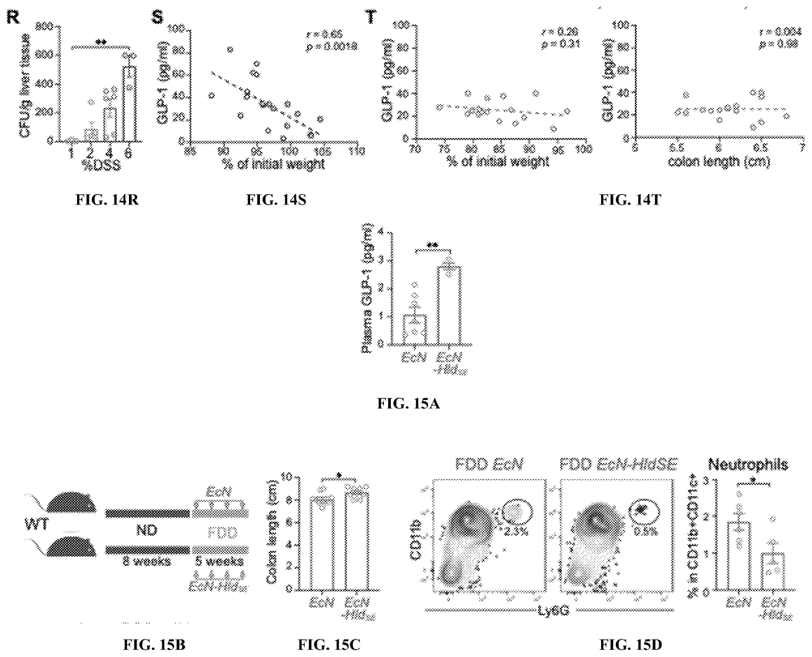

Diet-induced dysbiosis was explored in two IBD mouse models: acute DSS colitis and chronic Il10^{-/-} colitis. While ND-fed mice experienced only mild effects with 1.5% DSS, FDD-fed mice showed severe disease, with around 40% mortality, significant weight loss, and colon shortening, increased histological colitis score, along with increased gut permeability as indicated by higher CFU in the liver (FIGS. 14A-14F). Similarly, FDD worsened chronic colitis in Il10^{-/-} mice, leading to weight loss, colon shortening, liver CFU increase, and reduced apical junction gene expression after five weeks (FIGS. 14H-14L). Both models exhibited elevated neutrophils in the colon lamina propria (FIGS. 14G-14M). Overall, diet-mediated colitis consistently correlated with severe gut barrier dysfunction. Notably, this effect was at least partially attributed to the absence of SCFAs, since supplementing butyrate in the drinking water of FDD-fed mice eased colitis severity (FIGS. 14N-14P).

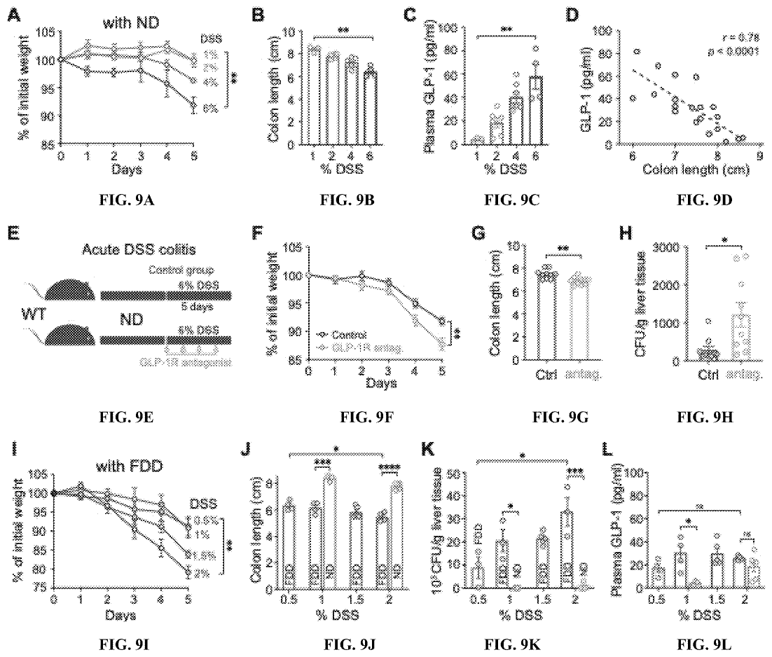

GLP-1 levels dynamically increased with the severity of tissue damage. With increasing DSS concentrations, colitis severity worsened in ND-fed mice, as evidenced by weight loss and colon shortening, as well as bacterial dissemination into liver (FIGS. 9A-9B, 14R). Circulating GLP-1 levels correspondingly rose with increased colitis severity (FIG. 9C), showing a significant correlation with colon length and weight loss (FIGS. 9D and 14S). Blocking GLP-1 receptor signaling during DSS colitis with a GLP-1 receptor antagonist (ANT) exacerbated disease severity, leading to more weight loss, greater colon shortening, and higher bacterial dissemination into the liver (FIGS. 9E-9H). These findings suggest that GLP-1 release is crucial for the initial mucosal defense against injury and bacterial invasion. However, in FDD-fed mice, GLP-1 release in response to tissue damage during DSS colitis was impaired. Even with increased DSS levels and severe colitis (FIGS. 91 and 9J), there was no substantial rise in the L-cell-derived peptide hormone GLP-1 (FIG. 9K). In the absence of dietary fiber, GLP-1 levels did not correlate with colitis severity (FIG. 14T), indicating that L-cell function was dysregulated under inflammatory conditions in FDD-fed mice. This dysregulation of GLP-1 could contribute significantly to the increased susceptibility to colitis observed in FDD-fed mice.

### Example 4

### GLP-1 Receptor Signaling Improves Colitis Symptoms in Dysbiotic Mice

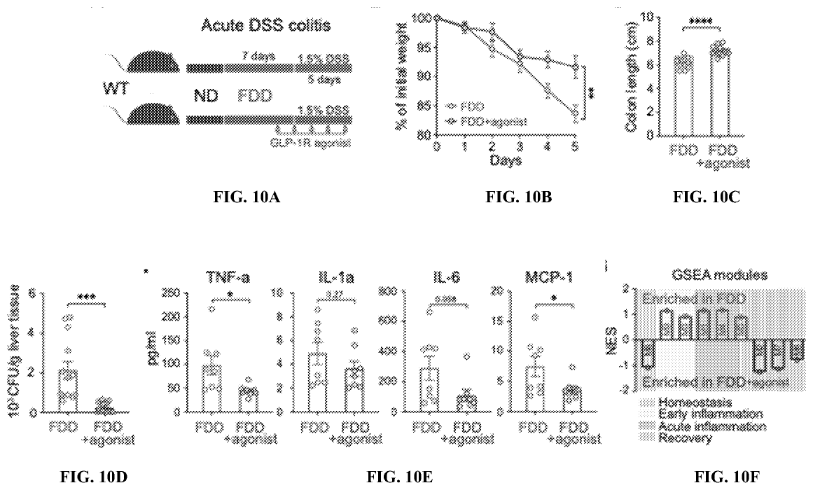

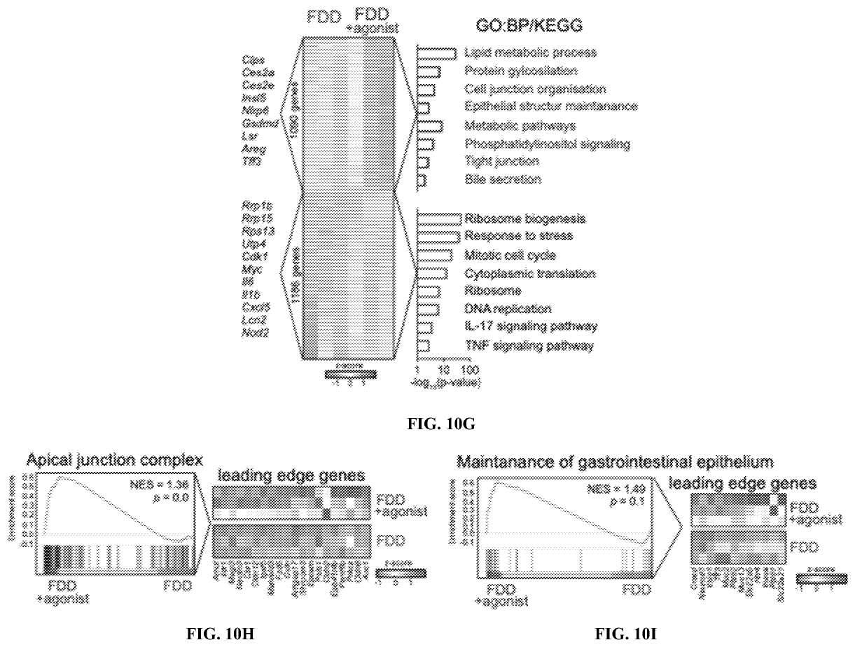

Treatment with a GLP-1 receptor agonist during DSS colitis (FIG. 10A) led to reduced weight loss and less colon shortening in FDD mice (FIGS. 10B-10C). Histological colitis score also improved with GLP-1 receptor agonist treatment. Restoration of GLP-1 signaling significantly reduced CFU levels in the liver, indicating enhanced gut barrier function (FIG. 10D), and was accompanied by lower circulating inflammatory cytokines, especially TNF-α and MCP-1 (FIG. 10E).

RNA-sequencing of colonic tissue from GLP-1 receptor agonist-treated FDD mice revealed a transcriptional shift during acute colitis. Using a time-series dataset of DSS colitis, gene set enrichment analysis (GSEA) revealed enrichment of genes associated with homeostasis and recovery phases (FIG. 10F). Over-representation analysis indicated upregulation of genes involved in lipid metabolic processes (e.g. Mlxipl, Ces2a, Ces2b, Cyp4f40, Clps), tight junctions and epithelial structure maintenance (e.g. Ocln, Clan8, Marveld2, Marveld3, Lsr, Neurod1) and bile secretion (e.g. Abcc3, Aqp4, Abcbla) in response to GLP-1 receptor agonist (FIG. 10G). Conversely, genes linked to ribosome biogenesis (e.g. Rrs1, Rrp12, Rcl1), cell cycle and DNA replication (e.g. Myc, Ccne1, Tuba8, Mcm2, Mcm3) and inflammatory signaling (e.g. S100a8, S100a9, Il6, Lcn2, Il1b, Mmp3, Mmp9) were downregulated in GLP-1 receptor agonist treated FDD mice during colitis (FIG. 10G). In agreement with these results, GSEA with GO terms identified GO: CC ‘apical junction complex’ and GO: BP ‘maintenance of gastrointestinal epithelium’ were amongst top enriched pathways in response to GLP-1 receptor agonist treatment (FIGS. 10H and 10I). Amongst leading edge genes influencing GO: BP ‘maintenance of gastrointestinal epithelium’ were notable genes involved in mucosal defense after injury such as goblet cell-released mucins (Muc2, Muc13) and trefoil factors (Tff3) (FIG. 10G). Key genes driving the enrichment in ‘apical junction complex’ included tight junction components (e.g., Ocln, Cldn family, Tjp family, Marveld3, Lsr) and adhesion molecules (e.g., Epcam, Ank3, Afdn, Cdh1). Maintaining tight junction integrity, which is often disrupted during colitis, is critical for preventing gut inflammation and promoting healing. Overall, GLP-1 signaling in FDD mice enhanced gut barrier function, reduced systemic inflammation, and facilitated epithelial recovery, emphasizing its potential role in strengthening mucosal defenses during colitis.

### Example 5

### Diet-Mediated Colitis is Ameliorated by an Engineered Probiotic in GLP-1 Dependent Manner

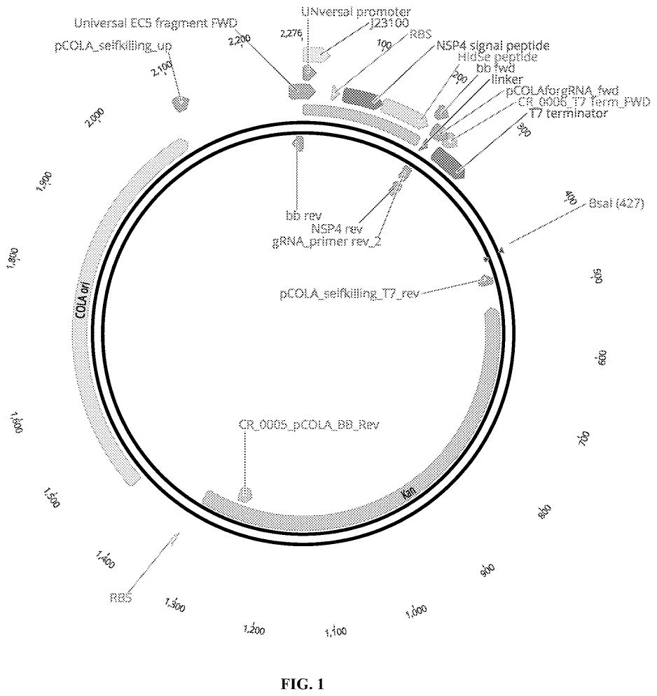

An engineered probiotic strategy was explored to potentially modulate GLP-1 levels locally in the intestine. In a screen of bacterial products that can stimulate gut peptides release, the 8-toxin (Hld_{SE}), a 25-amino acid peptide (MAADIISTIGDLVKWIIDTVNKFKK; SEQ ID NO: 1) from Staphylococcus epidermidis, was shown to induce GLP-1 release in vitro. The probiotic Escherichia coli Nissle 1917 (EcN) was engineered to produce Hld_{SE} (EcN-Hld_{SE}) and peptide secretion was enhanced using the NSP4 secretion tag (Han, S., et al., AMB Express, 2017. 7 (1): p. 93.) (FIG. 1).

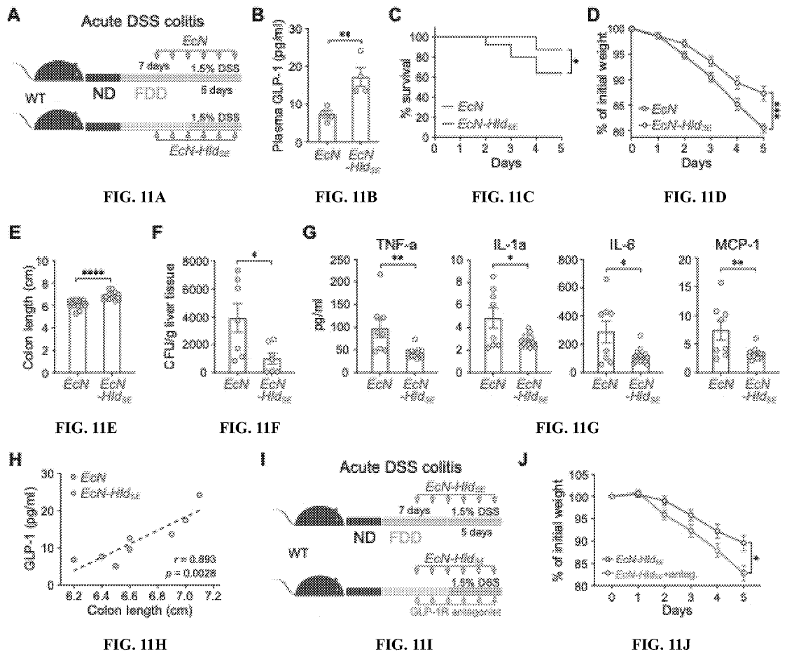

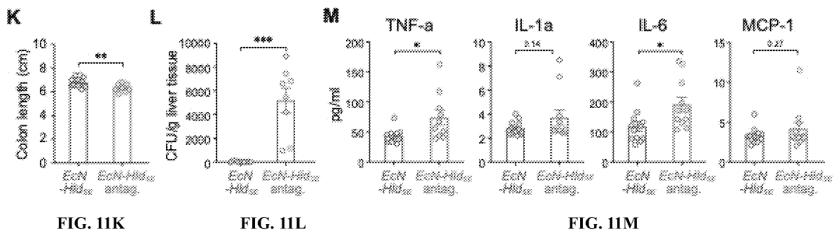

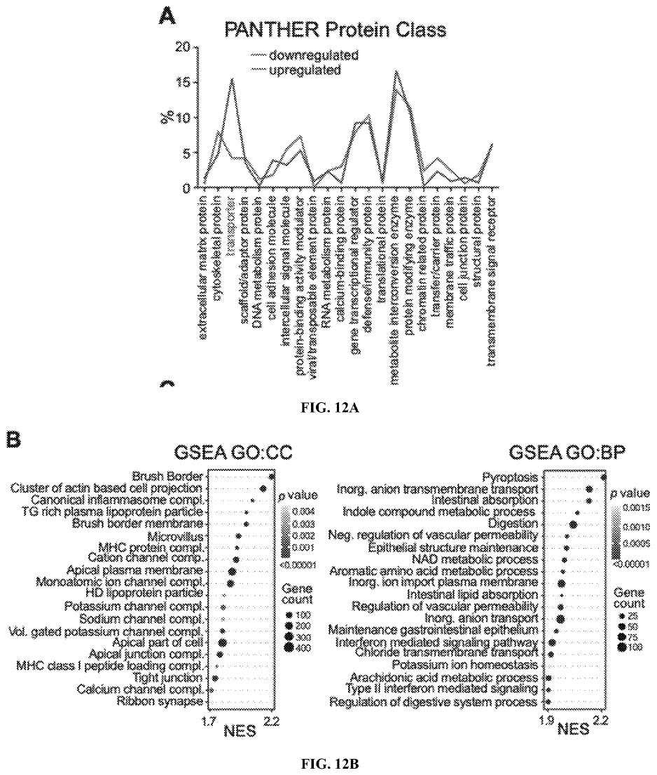

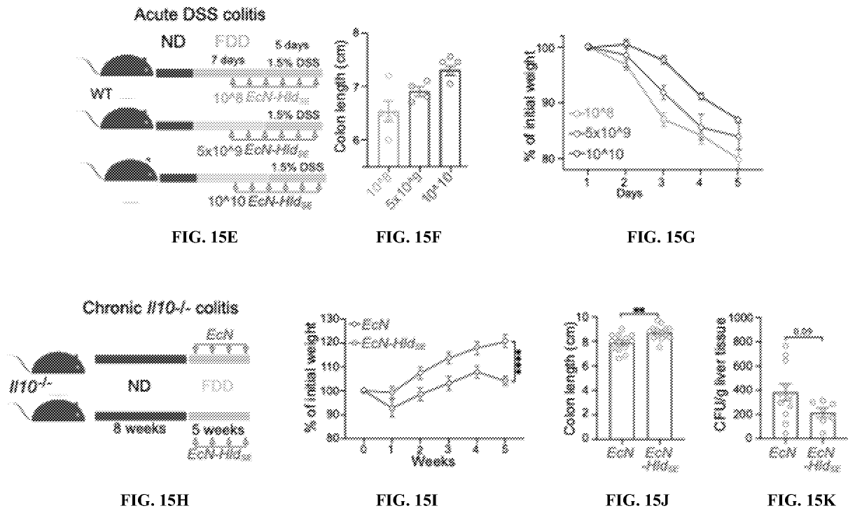

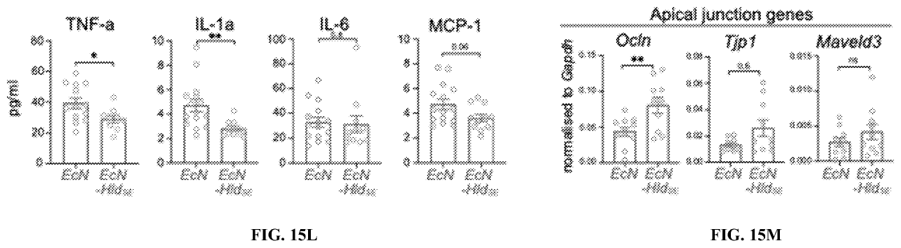

Strikingly, FDD mice orally gavaged with EcN-Hld_{SE} daily for one week showed a significant increase in circulating GLP-1 (FIG. 15A). Chronic gavage with EcN-Hld_{SE} for five weeks blunted the colon shortening and neutrophil infiltration into the colon lamina propria previously observed with FDD-feeding (FIGS. 15B-15D, 8C, 12B). During acute DSS colitis, daily treatment of FDD mice with EcN-Hld_{SE} also significantly increased circulating GLP-1 (FIGS. 11A-11B), improved survival, reduced weight loss, and reduced colon shortening (FIGS. 11C-11E). EcN-Hld_{SE} decreased bacteria dissemination into the liver and circulating pro-inflammatory cytokines, suggesting improved gut barrier function and lower systemic disease burden (FIGS. 11F-11G). Colon length positively correlated with GLP-1 levels, indicating that GLP-1 restoration contributed to at least some of these benefits (FIG. 11H). Of note, the therapeutic effect of EcN-Hld_{SE} was dose-dependent (FIGS. 15E-15G) and also extended to an FDD-fed Il10^{-/-} colitis mouse model, which showed improved weight, colon shortening, reduced systemic inflammation, and increased expression of apical junction proteins (FIGS. 15H-15M).

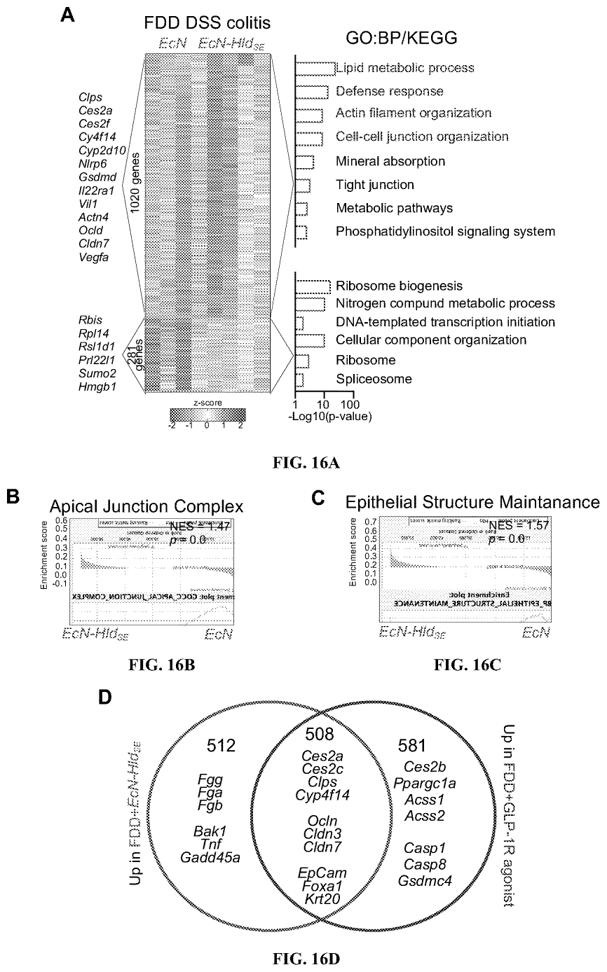

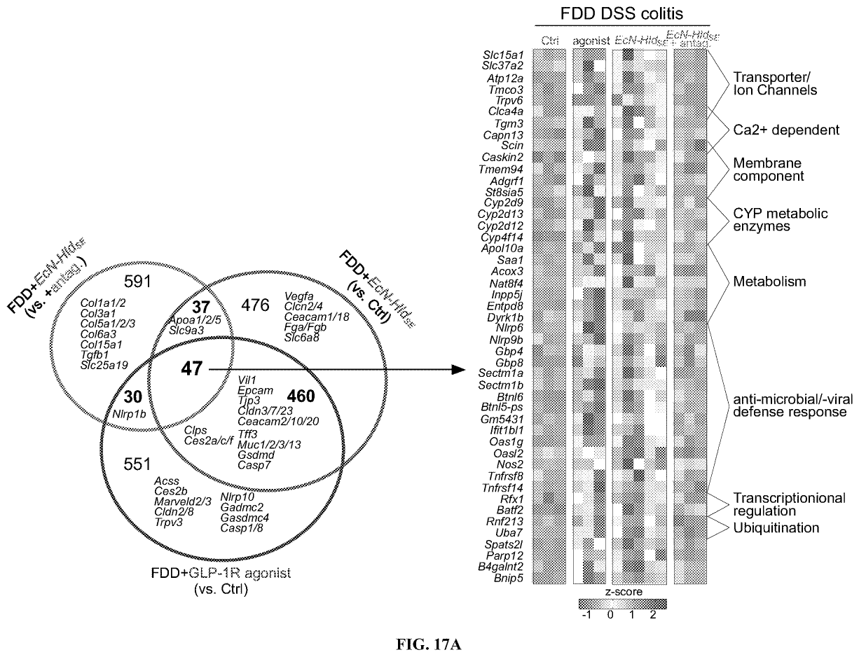

Over-representation analysis (ORA) from RNA-seq of colon tissue from FDD-fed mice treated with EcN-Hld_{SE} during DSS colitis showed an upregulation of genes involved in lipid metabolism (e.g.Clps, Apoa5, Ces2a, Ces2f, Cyp4f40), tight junctions (e.g. Marveld3, Ocln, Cldn7, Tjp3) and epithelial structure maintenance (e.g. Lsr, Muc2, Muc13, Tff3), similar to changes observed in FDD-fed mice treated with GLP-1 receptor agonist (FIG. 16A). Consistently, amongst top enriched GO terms in GSEA were apical junction complex and epithelial maintenance (FIGS. 16B-16C). Overall, nearly 50% of DEGs were commonly upregulated in the colon of mice treated with EcN-Hld_{SE} and the GLP-1 receptor agonist (FIG. 16D).

To further demonstrate the functional dependency of EcN-Hld_{SE} treatment on GLP-1 signaling, GLP-1 receptor signaling was blocked using a GLP-1 receptor antagonist (ANT) (FIG. 11I). GLP-1 receptor antagonism abolished protective effects of the engineered probiotic. Mice showed severe weight loss and shorter colons (FIGS. 11J-11K), as well as drastically increased number of bacteria in the liver and elevated level of circulating pro-inflammatory cytokines (FIGS. 11L-11M). These results demonstrated that EcN-Hld_{SE} induced GLP-1 release and mediated protective effects during colitis through GLP-1 receptor signaling.

### Example 6

### Transcriptional Changes in the Gut in Response to GLP-1 Receptor Signaling

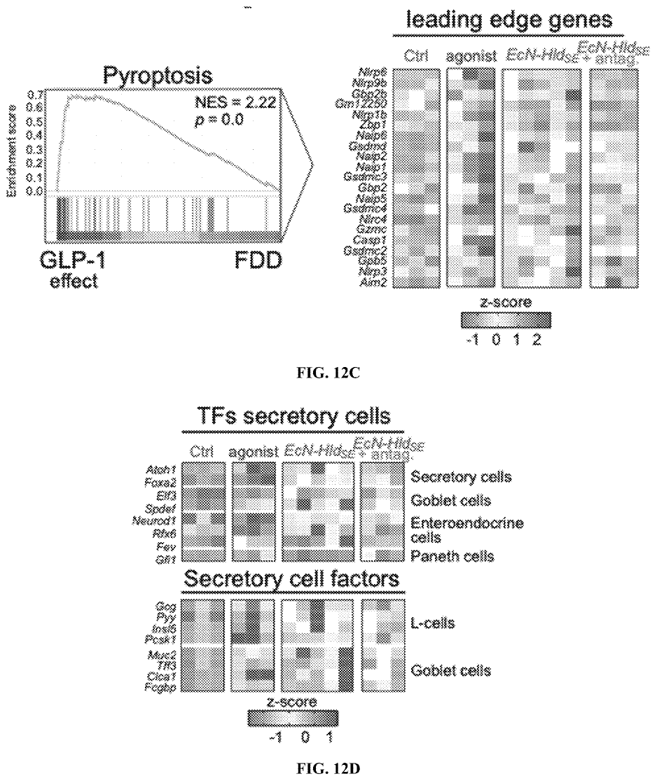

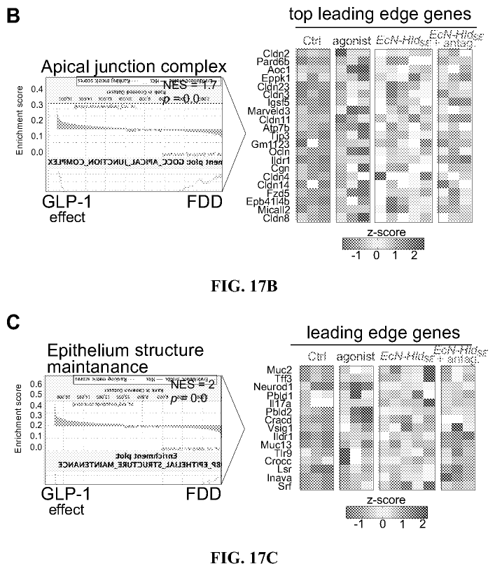

To validate the core changes induced by GLP-1 receptor signaling, the colonic transcriptional changes during DSS colitis were compared between FDD control mice, GLP-1 receptor agonist-treated mice, EcN-Hld_{SE}-treated mice, and mice treated with EcN-Hld_{SE} and a GLP-1 receptor antagonist. Shared upregulated DEGs were identified and a ranked gene list was generated based on the average log 2 fold changes of each gene in the three comparisons (FIG. 17A). The shared upregulated DEGs in response to GLP-1 modulating treatment during colitis primarily encoded transmembrane transporters and ion channels, metabolism-related genes, and genes involved in antimicrobial and antiviral defense (FIG. 17A). Analysis of top upregulated (average log 2 fold change >1; 743 genes) and downregulated (average log 2 fold change <−1; 299 genes) genes revealed substantial upregulation of ion channels and transporters (e.g., Slc26a3, Slc5a8, Clcn2, Trpv6, Kcnh2), contributing to enrichment in GO pathways related to ion channel complexes, colonic brush border, microvilli, and apical plasma membrane (FIGS. 12A-12B). Enrichment of GO: CC ‘tight junctions’ and GO: BP ‘maintenance of gastrointestinal epithelium’ was in line with previous analyses (FIGS. 12B, 17B-17C). Additionally, GLP-1 signaling improved intestinal digestion and absorption, particularly in genes associated with vitamin and lipid absorption (e.g., Isx, Abcg5, Apoa1, Cyp8b1) as well as cellular metabolism, such as NAD metabolism (e.g., Apoa2, Idol, Nadsyn1, Cyp2d12, Haao) (FIG. 12B). Notably, Nlrp6, essential for gut homeostasis by promoting mucus and antimicrobial peptide secretion and protection against colitis, emerged as a top leading-edge gene in the GO ‘proptosis’ pathway (FIGS. 12B and 12C).

Analysis of transcriptional regulators revealed enrichment of factors involved in intestinal secretory cell differentiation (e.g., Foxa2, Spdef, Atoh1, Neurod1) (FIG. 12D) and metabolic regulation (e.g., Hnf4g, Hnf4a, Ppargc1a, Vdr) in response to GLP-1. Downregulated transcription factors were related to cell proliferation, renewal (e.g., E2f8, Mxd3, Hmga2), and inflammation (e.g., Nfe2, Ifi205, Spic). In line with these findings, L-cell-related genes (Gcg, Pyy, Ins15, Pcsk1) and goblet cell-related genes (Muc2, Tff3, Clca1, Fcgbp), both secretory cell subsets, were upregulated, suggesting that GLP-1 can enhance intestinal secretory cell types, as previously indicated by in vitro findings. In accord, an increase in L-cell numbers were observed in GLP-1 receptor agonist-treated FDD mice during DSS colitis; goblet cell numbers remained unchanged (FIG. 12E). Strikingly, using an extensive dataset of human colon biopsies, genes encoding transcription factors driving enteroendocrine differentiation (Neurod1, Rfx6, Fev) and L-cell hormones (Gcg, Pyy, Insl5) were downregulated in IBD patients, even during disease remission (FIG. 12F), suggesting potential benefits of GLP-1 modulation therapy for IBD patients. Overall, GLP-1 receptor signaling enhances the expression of genes involved in key functions of the gut including ion transport, absorption of vitamins and nutrients, metabolism, and barrier function. Additionally, GLP-1 signaling increased genes involved in intestinal secretory cell differentiation, particularly in L-cells and influenced factors involved in protective mucus secretion.

The scope of the present invention is not limited by what has been specifically shown and described hereinabove. Those skilled in the art will recognize that there are suitable alternatives to the depicted examples of materials, configurations, constructions, and dimensions. Variations, modifications, and other implementations of what is described herein will occur to those of ordinary skill in the art without departing from the spirit and scope of the invention.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

1. A bacterium engineered for the production of a heterologous delta hemolysin peptide or variant or polypeptide comprising thereof,
wherein the bacterium comprises a nucleic acid encoding the delta hemolysin peptide or variant or polypeptide comprising thereof.

2. The bacterium of claim 1, wherein the nucleic acid is incorporated into the host genome or episomal.

3. The bacterium of claim 1, wherein the delta hemolysin peptide is derived from a Staphylococcal species.

4. The bacterium of claim 1, wherein the delta hemolysin peptide has an amino acid sequence of MAADIISTIGDL VKWIIDTVNKFKK (SEQ ID NO: 1).

5. The bacterium of claim 1, wherein the delta hemolysin peptide is linked to a signal peptide.

6. The bacterium of claim 1, wherein the bacterium is a non-pathogenic bacterium, a commensal bacterium, or a probiotic bacterium.

7. The bacterium of claim 1, wherein the bacterium is from the genus Escherichia.

8. The bacterium of claim 1, wherein the bacterium is a strain of gut bacteria.

9. A composition comprising a bacterium of claim 1, or a population thereof.

10. The composition of claim 9, wherein the composition is food or beverage product.

11. A method for improving or maintaining gut health, regulating glucose metabolism, maintaining intestinal integrity, and/or promoting tissue repair and regeneration comprising administering to a subject in need a bacterium of claim 1, or a population or composition comprising thereof, or a nucleic acid encoding a delta hemolysin peptide.

12. A method for improving or maintaining gut health, regulating glucose metabolism, maintaining intestinal integrity, and/or promoting tissue repair and regeneration in a subject in need thereof comprising introducing a nucleic acid encoding a delta hemolysin peptide or variant or polypeptide thereof into a bacterium in the subject.

13. The method of claim 11, wherein the delta hemolysin peptide has an amino acid sequence of MAADIISTIGDL VKWIIDTVNKFKK (SEQ ID NO: 1).

14. The method of claim 11, wherein the subject has or is suspected of having an inflammatory bowel disease, selected from Crohn's disease and ulcerative colitis.

15. (canceled)

16. The method of claim 12, wherein the delta hemolysin peptide has an amino acid sequence of MAADIISTIGDLVKWIIDTVNKFKK (SEQ ID NO: 1).

17. The method of claim 12, wherein the subject has or is suspected of having an inflammatory bowel disease, selected from Crohn's disease and ulcerative colitis.
